(19) United States
(12) Patent Application Publication
(10) Pub. No.: US 2006/0003952 A1
(43) Pub. Date: Jan. 5, 2006

(54) Oligomeric compounds having modified phosphate groups

(75) Inventors: Vasulinga Ravikumar, Carlsbad, CA (US); Thazha R Prakash, Carlsbad, CA (US); Balkrishen Bhat, Carlsbad, CA (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: ISIS Pharmaceuticals, Inc., Carlsbad, CA (US)

(21) Appl. No.: 10/510,667

(22) PCT Filed: Apr. 9, 2003
(86) PCT No.: PCT/US03/10840

§ 371 Date: Apr. 5, 2005

(30) Foreign Application Priority Data
Apr. 9, 2002 (US) 10/119,432

(51) Int. Cl. A61K 48/00; C07H 21/02

(52) U.S. Cl. 514/44; 536/23.1

(57) ABSTRACT

Oligomeric compounds having at least one phosphorothioate monoester are provided having increased nuclease resistance and binding affinity to a complementary strand of nucleic acid. Such oligomeric compounds are useful for diagnostics and other research purposes, for modulating the expression of a protein in organisms, and for the diagnosis, detection and treatment of other conditions responsive to oligonucleotide therapeutics.

### FIELD OF THE INVENTION

The present invention relates to oligomeric compounds having at least one modified phosphate group. The oligomeric compounds of the present invention typically have enhanced RNase H activation properties compared to oligomeric compounds without the modification. The oligomeric compounds are useful for investigative and therapeutic purposes.

### BACKGROUND OF THE INVENTION

It is well known that most of the bodily states in mammals, including most disease states, are affected by proteins. Classical therapeutic modes have generally focused on interactions with such proteins in an effort to moderate their disease-causing or disease-potentiating functions. Recently, however, attempts have been made to moderate the actual production of such proteins by interactions with molecules that direct their synthesis, such as intracellular RNA. By interfering with the production of proteins, maximum therapeutic effect and minimal side effects may be realized. It is the general object of such therapeutic approaches to interfere with or otherwise modulate gene expression leading to undesired protein formation.

One method for inhibiting specific gene expression is the use of oligonucleotides. Oligonucleotides are now accepted as therapeutic agents with great promise. Oligonucleotides are known to hybridize to single-stranded DNA or RNA molecules. Hybridization is the sequence-specific base pair hydrogen bonding of nucleobases of the oligonucleotide to the nucleobases of the target DNA or RNA molecule. Such nucleobase pairs are said to be complementary to one another. The concept of inhibiting gene expression through the use of sequence-specific binding of oligonucleotides to target RNA sequences, also known as antisense inhibition, has been demonstrated in a variety of systems, including living cells. See, Wagner et al., Science (1993) 260: 1510-1513; Milligan et al., J. Med. Chem., (1993) 36:1923-37; Uhlmann et al., Chem. Reviews, (1990) 90:543-584; Stein et al., Cancer Res., (1988) 48:2659-2668.

Events that provide disruption of the nucleic acid function by antisense oligonucleotides (Cohen in Oligonucleotides: Antisense Inhibitors of Gene Expression, (1989) CRC Press, Inc., Boca Raton, Fla.) are thought to be of two types. The first, hybridization arrest, denotes the terminating event in which the oligonucleotide inhibitor binds to the target nucleic acid and thus prevents, by simple steric hindrance, the binding of essential proteins, most often ribosomes, to the nucleic acid. Methyl phosphonate oligonucleotides (Miller and Ts′O, Anti-Cancer Drug Design, 1987, 2:117-128) and α-anomer oligonucleotides are the two most extensively studied antisense agents which are thought to disrupt nucleic acid function by hybridization arrest.

The second type of terminating event for antisense oligonucleotides involves the enzymatic cleavage of the targeted RNA by intracellular RNase H. A 2′-deoxyribofuranosyl oligonucleotide or oligonucleotide analog hybridizes with the targeted RNA and this duplex activates the RNase H enzyme to cleave the RNA strand, thus destroying the normal function of the RNA. Phosphorothioate oligonucleotides are the most prominent example of an antisense agent that operates by this type of antisense terminating event.

Oligonucleotides may also bind to duplex nucleic acids to form triplex complexes in a sequence specific manner via Hoogsteen base pairing (Beal et al., Science, (1991) 251:1360-1363; Young et al., Proc. Natl. Acad. Sci. (1991) 88:10023-10026). Both antisense and triple helix therapeutic strategies are directed towards nucleic acid sequences that are involved in or responsible for establishing or maintaining disease conditions. Such target nucleic acid sequences may be found in the genomes of pathogenic organisms including bacteria, yeasts, fungi, protozoa, parasites, viruses, or may be endogenous in nature. By hybridizing to and modifying the expression of a gene important for the establishment, maintenance or elimination of a disease condition, the corresponding condition may be cured, prevented or ameliorated.

In determining the extent of hybridization of an oligonucleotide to a complementary nucleic acid, the relative ability of an oligonucleotide to bind to the complementary nucleic acid may be compared by determining the melting temperature of a particular hybridization complex. The melting temperature (Tₘ), a characteristic physical property of double helices, denotes the temperature (in degrees centigrade) at which 50% helical (hybridized) versus coil (unhybridized) forms are present. Tₘ is measured by using the UV spectrum to determine the formation and breakdown (melting) of the hybridization complex. Base stacking, which occurs during hybridization, is accompanied by a reduction in UV absorption (hypochromicity). Consequently, a reduction in UV absorption indicates a higher Tₘ. The higher the Tₘ, the greater the strength of the bonds between the strands.

Oligonucleotides may also be of therapeutic value when they bind to non-nucleic acid biomolecules such as intracellular or extracellular polypeptides, proteins, or enzymes. Such oligonucleotides are often referred to as “aptamers” and they typically bind to and interfere with the function of protein targets (Griffin et al., Blood, (1993), 81:3271-3276; Bock et al., Nature, (1992) 355: 564-566).

Oligonucleotides and their analogs have been developed and used for diagnostic purposes, therapeutic applications and as research reagents. For use as therapeutics, oligonucleotides must be transported across cell membranes or be taken up by cells, and appropriately hybridize to target DNA or RNA. These critical functions depend on the initial stability of the oligonucleotides toward nuclease degradation. A serious deficiency of unmodified oligonucleotides which affects their hybridization potential with target DNA or RNA for therapeutic purposes is the enzymatic degradation of administered oligonucleotides by a variety of intracellular and extracellular ubiquitous nucleolytic enzymes referred to as nucleases. For oligonucleotides to be useful as therapeutics or diagnostics, the oligonucleotides should demonstrate enhanced binding affinity to complementary target nucleic acids, and preferably be reasonably stable to nucleases and resist degradation. For a non-cellular use such as a research reagent, oligonucleotides need not necessarily possess nuclease stability.

A number of chemical modifications have been introduced into oligonucleotides to increase their binding affinity to target DNA or RNA and increase their resistance to nuclease degradation.

Modifications have been made to the ribose phosphate backbone of oligonucleotides to increase their resistance to nucleases. These modifications include use of linkages such as methyl phosphonates, phosphorothioates and phosphorodithioates, and the use of modified sugar moieties such as 2′-O-alkyl ribose. Other oligonucleotide modifications include those made to modulate uptake and cellular distribution. A number of modifications that dramatically alter the nature of the internucleotide linkage have also been reported in the literature. These include non-phosphorus linkages, peptide nucleic acids (PNA's) and 2′-5′ linkages. Another modification to oligonucleotides, usually for diagnostic and research applications, is labeling with non-isotopic labels, e.g., fluorescein, biotin, digoxigenin, alkaline phosphatase, or other reporter molecules.

A variety of modified phosphorus-containing linkages have been studied as replacements for the natural, readily cleaved phosphodiester linkage in oligonucleotides. In general, most of them, such as the phosphorothioate, phosphoramidates, phosphonates and phosphorodithioates all result in oligonucleotides with reduced binding to complementary targets and decreased hybrid stability. In order to make effective therapeutics therefore this binding and hybrid stability of antisense oligonucleotides needs to be improved.

Of the large number of modifications made and studied, few have progressed far enough through discovery and development to deserve clinical evaluation. Reasons underlying this include difficulty of synthesis, poor binding to target nucleic acids, lack of specificity for the target nucleic acid, poor in vitro and in vim stability to nucleases, and poor pharmacokinetics. Several phosphorothioate oligonucleotides and derivatives are presently being used as antisense agents in human clinical trials for the treatment of various disease states. Approval to use the antisense drug, Fomivirsen, to treat cytomegalovirus (CMV) retinitis in humans was recently granted by both the United States and European regulatory agencies.

The structure and stability of chemically modified nucleic acids is of great importance to the design of antisense oligonucleotides. Over the last ten years, a variety of synthetic modifications have been proposed to increase nuclease resistance, or to enhance the affinity of the antisense strand for its target mRNA (Crooke et al., Med. Res. Rev., 1996, 16, 319-344; De Mesmaeker et al., Acc. Chem. Res., 1995, 28, 366-374). Although a great deal of information has been collected about the types of modifications that improve duplex formation, little is known about the structural basis for the improved affinity observed

RNA exists in what has been termed “A Form” geometry while DNA exists in “B Form” geometry. In general, RNA:RNA duplexes are more stable, or have higher melting temperatures (Tm) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). In addition, the 2′ hydroxyl groups of RNA can form a network of water mediated hydrogen bonds that help stabilize the RNA duplex (Egli et al., Biochemistry, 1996, 35, 8489-8494).

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of a DNA:RNA hybrid is central to antisense therapies as the mechanism requires the binding of a modified DNA strand to a mRNA strand. To effectively inhibit the mRNA, the antisense DNA should have a very high binding affinity with the mRNA. Otherwise the desired interaction between the DNA and target mRNA strand will occur infrequently, thereby decreasing the efficacy of the antisense oligonucleotide.

One synthetic 2′-modification that imparts increased nuclease resistance and a very high binding affinity to nucleotides is the 2′-methoxyethoxy (MOE, 2′-OCH₂CH₂OCH₃) side chain (Baker et al., J. Biol. Chem., 1997, 272, 11944-12000; Freier et al., Nucleic Acids Res., 1997, 25, 4429-4443). One of the immediate advantages of the MOE substitution is the improvement in binding affinity, which is greater than many similar 2′ modifications such as O-methyl, O-propyl, and O-aminopropyl (Freier and Altmann, Nucleic Acids Research, (1997) 25:4429-4443). Oligonucleotides and oligonucleotide analogs having 2′-O-methoxyethyl-substitutions have also been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al, Nucleosides Nucleotides, 1997, 16, 917-926). Relative to DNA, they display improved RNA affinity and higher nuclease resistance. Chimeric oligonucleotides with 2′-O-methoxyethyl-ribonucleoside wings and a central DNA-phosphorothioate window also have been shown to effectively reduce the growth of tumors in animal models at low doses. MOE substituted oligonucleotides have shown outstanding promise as antisense agents in several disease states. One such MOE substituted oligonucleotide is currently available for the treatment of CMV retinitis.

The conversion of alcohols to phosphate monoesters has been reported in Wada et al., Tetrahedron Letters, 1998, 39, 7123-7126.

The synthesis of oligonucleotides incorporating 2′-O-phosphorylated ribonucleotides has been reported in Tsuruoka et al., J. Org. Chem., 2000, 65, 7479-7494. They also report the synthesis of a deoxyuridylate 10 mer wherein an intermediate to the final 2′-phosphorylated 10 mer is a 2′-phosphorothioate monoester function on the 6 position of the deoxyoligonucleotide while still attached to a solid support.

The synthesis of N-phosphorylated ribonucleosides has been reported in Wada et al., J. Am. Chem. Soc., 1994, 116, 9901-9911.

U.S. Pat. No. 6,033,909 to Uhlmann et al. discloses modified phosphorothioate oligonucleotides. Roland et al., Tetrahedron Letters, 2001, 42, 3669-3672, disclose the use of controlled pore glass (CPG) support with an acyloxyaryl group as a linker to make libraries of small molecules of 3′-thiophosphorylated dinucleotides by solid-phase synthesis. Alefelder, et al., Nucleic Acids Research, (1998) 26:4983-4988, disclose a method to introduce terminal phosphorothioates on only the 3′ or 5′ ends for further derivatization.

In another recently published paper (Martinez et al., Cell, 2002, 110, 563-574) it was shown that double stranded as well as single stranded siRNA resides in the RNA-induced silencing complex (RISC) together with elF2C1 and elf2C2 (human GERp950 Argonaute proteins. The activity of 5′-phosphorylated single stranded siRNA was comparable to the double stranded siRNA in the system studied. In a related study, the inclusion of a 5′-phosphate moiety was shown to enhance activity of siRNA's in vivo in Drosophilia embryos (Boutla, et al., Curr. Biol., 2001, 11, 1776-1780). In another study, it was reported that the 5′-phosphate was required for siRNA function in human HeLa cells (Schwarz et al., Molecular Cell, 2002, 10, 537-548).

As described above, the versatility of phosphorothioate ester modifications is limited. Although the known modifications to oligonucleotides, including the use of the 2′-O-methoxyethyl modification, have contributed to the development of oligonucleotides for various uses, there still exists a need in the art for further modifications that offer the opportunity for enhanced hybrid binding affinity and/or increased nuclease resistance.

### SUMMARY OF THE INVENTION

In accordance with one embodiment of the present invention there are provided oligomeric compounds of the formula: 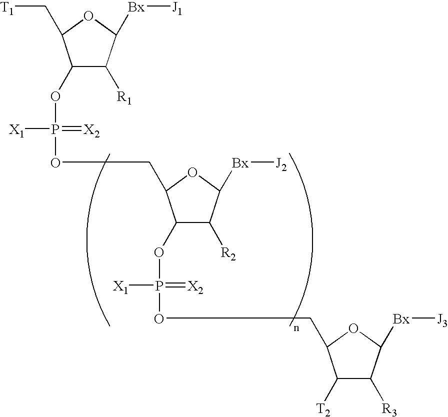
wherein:
each Bx is, independently, a heterocyclic base moiety;
J₁, J₃ and each J₂ is, independently, hydrogen or a modified phosphate group having the structure: 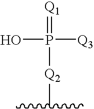
wherein
   one of Q₁ and Q₂ is S and the other of Q₁ and Q₂ is O;
   Q₃ is OH or CH₃;
   R₁, R₃ and each R₂ is, independently, hydrogen, hydroxyl, a sugar substituent group a protected sugar substituent group or said modified phosphate group;
each T₁ and T₂ is, independently, hydroxyl, a protected hydroxyl, an oligonucleotide, an Oligonucleoside or said modified phosphate group;
each X₁ and X₂ is, independently, O or S wherein at least one X₁ is S;
n is from 3 to 48; and
wherein at least one of J₁, J₂, J₃, R₁, R₂, R₃, T₁ or T₂ is said modified phosphate group.

Some of the oligomeric compounds of this invention have Q₁ as S. In other oligomeric compounds Q₂ is S.

In some of the oligomeric compounds of this invention Q₃ is CH₃. In other oligomeric compounds Q₃ is OH.

In one embodiment of this invention J₁ is a modified phosphate group. In other embodiments, at least one J₂ is a modified phosphate group. In further embodiments J₃ is a modified phosphate group.

In one embodiment of this invention R₁ is a modified phosphate group. In other embodiments, a least one R₂ is a modified phosphate group. In further embodiments R₃ is a modified phosphate group.

In one embodiment of this invention R₁, R₃ and each R₂ is hydrogen In a further embodiment R₁, R₃ and each R₂ is hydroxyl. And in a further embodiment R₁, R₃ and each R₂ is hydrogen, hydroxyl a sugar substituent group or a protected sugar substituent group. In a further embodiment at least one of R₁, R₂ or R₃ is an optionally protected sugar substituent group.

In one embodiment of the present invention each X₂ is S.

Embodiments of this invention can exist wherein each heterocyclic base moiety is, independently, adenine, cytosine, 5-methylcytosine, thymine, uracil, guanine or 2-aminoadenine. The variable n can be from about 8 to about 30 with about 15 to 25 being preferred.

The present invention also provides methods for treating an organism having a disease characterized by the undesired production of an protein. These methods include contacting the organism with one or more of the above-noted oligomeric compounds.

Also provided are compositions including a pharmaceutically effective amount of an oligomeric compound of the invention and a pharmaceutically acceptable diluent or carrier.

The invention also provides methods for in vitro modification of a nucleic acid, including contacting a test solution containing an RNase H enzyme and the nucleic acid with an oligomeric compound of the invention.

In a further aspect, the invention provides methods of concurrently enhancing hybridization and RNase H enzyme activation in an organism that include contacting the organism with an oligomeric compound of the invention.

In yet a further embodiment of this invention, methods are provided comprising contacting a cell with an oligomeric compound of the invention.

### DETAILED DESCRIPTION OF PREFERRED EMBODIMENTS

The present invention provides modified oligomeric compounds useful in the regulation of gene expression. More specifically the oligonucleotides of the invention modulate gene expression by an antisense mechanism that includes RNAse H and RNA interference pathways. The oligonucleotides of the invention are modified to have modified phosphate groups. Preferred modified phosphate groups according to the present invention include without limitation, phosphorothioate monoesters and methyl phosphorothionates. In one embodiment the oligomeric compounds of this invention have enhanced R-Nase H activation properties as compared to similar unmodified oligomeric compounds.

By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art.

An oligomeric compound having the formula: 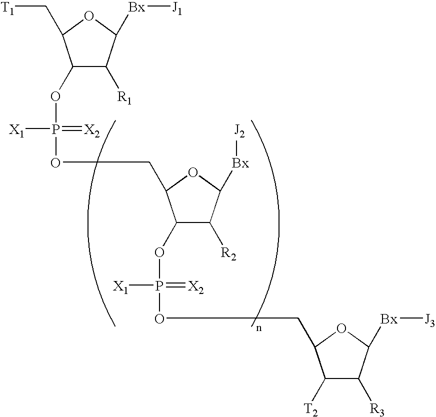
wherein:
each Bx is, independently, a heterocyclic base moiety;
J₁, J₃ and each J₂ is, independently, hydrogen or a modified phosphate group;
R₁, R₃ and each R₂ is, independently, H, an optionally protected sugar substituent group or a modified phosphate group;
each T₁ and T₂ is, independently, hydroxyl a protected hydroxyl, an oligonucleotide, an oligonucleoside or a modified phosphate group;
each X₁ and X₂ is, independently, O or S wherein at least one X₁ is S;
n is from 3 to 48; and
wherein at least one of J₁, J₂, J₃, R₁, R₂, R₃, T₁ or T₂ is a modified phosphate group.

The oligomeric compounds of the present invention comprise covalently linked nucleosidic monomers with at least one of the monomers having a modified phosphate group covalently attached thereto. Modified phosphate groups can be covalently attached to any nucleosidic monomer comprising an oligomeric compound of the invention, however the preferred point of attachment is to a 3′ or 5′-terminal monomer. The site of attachment on a selected nucleosidic monomer is also variable with 2′, 3′, or 5′-sugar hydroxyl groups and functional groups on the heterocyclic base moiety, such as an amino groups, all viable sites.

The oligomeric compounds of the invention can also be prepared using various chemistries known in the art to produce various internucleoside linkages. Uniform as well as mixed backbone oligomers are amenable to the present invention. Preferred internucleoside linkages include phosphorotioate and phosphorodithioate linkages. Preferred mixed backbone oligomers include those having phosphorothioate and phosphodiester internucleoside linkages.

The oligomeric compounds of the invention are useful for identification or quantification of an RNA or DNA or for modulating the activity of an RNA or DNA molecule. The oligomeric compounds having a modified nucleosidic monomer therein are preferably prepared to be specifically hybridizable with a preselected nucleotide sequence of a single-stranded or double-stranded target DNA or RNA molecule. It is generally desirable to select a sequence of DNA or RNA which is involved in the production of a protein whose synthesis is ultimately to be modulated or inhibited in its entirety or to select a sequence of RNA or DNA whose presence, absence or specific amount is to be determined in a diagnostic test.

Nucleosidic monomers used to prepare oligomeric compounds of the invention routinely include appropriate activated phosphorus groups such as activated phosphate groups and activated phosphite groups. As used herein, the terms activated phosphate and activated phosphite groups refer to activated monomers or oligomers that react with a hydroxyl group of another monomeric or oligomeric compound to form a phosphorus-containing internucleotide linkage. Such activated phosphorus groups contain activated phosphorus atoms in P^{III} or P^{v} valency states. Such activated phosphorus atoms are known in the art and include, but are not limited to, phosphoramidite, H-phosphonate and phosphate triesters. A preferred synthetic solid phase synthesis utilizes phosphoramidites as activated phosphates. The phosphoramidites utilize P^{III} chemistry. The intermediate phosphite compounds are subsequently oxidized to the P^{V} state using known methods to yield, in preferred embodiments, phosphorothioate or mixed phosphodiester and phosphorothioate internucleotide linkages. Additional activated phosphates and phosphites are disclosed in Tetrahedron Report Number 309 (Beaucage and Iyer, Tetrahedron, 1992, 48, 2223-2311).

The oligomeric compounds of the invention are conveniently synthesized using solid phase methodologies, and are preferably designed to be complementary to or specifically hybridizable with a preselected nucleotide sequence of the target RNA or DNA. Standard solution phase and solid phase methods for the synthesis of oligomeric compounds are well known to those skilled in the art. These methods are constantly being improved in ways that reduce the time and cost required to synthesize these complicated compounds. Representative solution phase techniques are described in U.S. Pat. No. 5,210,264, issued May 11, 1993 and commonly assigned with this invention. Representative solid phase techniques employed for the synthesis of oligomeric compounds utilizing standard phosphoramidite chemistries are described in Protocols For Oligonucleotides And Analogs, S. Agrawal, ed., Humana Press, Totowa, N.J., 1993.

The oligomeric compounds of the invention also include those that comprise nucleosides connected by charged linkages and whose sequences are divided into at least two regions. In some preferred embodiments, the first region is linked by a first type of linkage, and the second region includes nucleosides linked by a second type of linkage. In other preferred embodiments, the oligomers of the present invention further include a third region comprised of nucleosides as are used in the first region, with the second region positioned between the first and the third regions. Such oligomeric compounds are known as “chimeras,” “chimeric,” or “gapped” oligomers (See, e.g., U.S. Pat. No. 5,623,065, issued Apr. 22, 1997, the contents of which are incorporated herein by reference).

Examples of chimeric oligonucleotides include but are not limited to “gapmers,” in which three distinct regions are present, normally with a central region flanked by two regions which are chemically equivalent to each other but distinct from the gap. A preferred example of a gapmer is an oligonucleotide in which a central portion (the “gap”) of the oligonucleotide serves as a substrate for RNase H and is preferably composed of 2′-deoxynucleotides, while the flanking portions (the 5′ and 3′ “wings”) are modified to have greater affinity for the target RNA molecule but are unable to support nuclease activity (e.g., 2′-fluoro- or 2′-O-methoxyethyl-substituted). Other chimeras include “wingmers,” also known in the art as “hemimers,” that is, oligonucleotides with two distinct regions. In a preferred example of a wingmer, the 5′ portion of the oligonucleotide serves as a substrate for RNase H and is preferably composed of 2′-deoxynucleotides, whereas the 3′ portion is modified in such a fashion so as to have greater affinity for the target RNA molecule but is unable to support nuclease activity (e.g., 2′-fluoro- or 2′-O-methoxyethyl-substituted), or vice-versa. In one embodiment, the oligonucleotides of the present invention contain a 2′-O-methoxyethyl (2′-O—CH₂CH₂OCH₃) modification on the sugar moiety of at least one nucleotide. This modification has been shown to increase both affinity of the oligonucleotide for its target and nuclease resistance of the oligonucleotide. According to the invention, one, a plurality, or all of the nucleotide subunits of the oligonucleotides of the invention may bear a 2′-O-methoxyethyl (—O—CH₂CH₂OCH₃) modification. Oligonucleotides comprising a plurality of nucleotide subunits having a 2′-O-methoxyethyl modification can have such a modification on any of the nucleotide subunits within the oligonucleotide, and may be chimeric oligonucleotides. Aside from or in addition to 2′-O-methoxyethyl modifications, oligonucleotides containing other modifications which enhance antisense efficacy, potency or target affinity are also preferred. Chimeric oligonucleotides comprising one or more such modifications are presently preferred. Through use of such modifications, active oligonucleotides have been identified which are shorter than conventional “first generation” oligonucleotides active against mdm2. Oligonucleotides in accordance with this invention are from 5 to 50 nucleotides in length, preferably from about 8 to about 30. In the context of this invention it is understood that this encompasses non-naturally occurring oligomers as hereinbefore described, having from 5 to 50 monomers, preferably from about 8 to about 30.

Gapmer technology has been developed to incorporate modifications at the ends (“wings”) of oligomeric compounds, leaving a phosphorothioate gap in the middle for RNase H activation (Cook, P. D., Anti-Cancer Drug Des., 1991, 6,585-607; Monia et al., J. Biol. Chem., 1993, 268, 14514-14522). In a recent report, the activities of a series of uniformly 2′-O modified 20 mer RNase H-independent oligonucleotides that were antisense to the 5′-cap region of human ICAM-1 transcript in HUVEC cells, were compared to the parent 2′-deoxy phosphorothioate oligonucleotide (Baker et al., J. Bio. Chem., 1997, 272, 11994-12000). The 2′-MOE/P′O oligomer demonstrated the greatest activity with a IC₅₀ of 2.1 nM (Tₘ=87.1° C.), while the parent P═S oligonucleotide analog had an IC₅₀ of 6.5 nM (Tₘ=79.2° C.). Correlation of activity with binding affinity is not always observed as the 2′-F/P═S (Tₘ=87.9° C.) was less active than the 2′-MOE/P═S (Tₘ=79.2° C.) by four fold. The RNase H competent 2′-deoxy P═S parent oligonucleotide exhibited an IC₅₀=41 nM.

In the context of this invention, the terms “oligomer” and “oligomeric compound” refer to a plurality of naturally-occurring or non-naturally-occurring nucleosides joined together in a specific sequence. The terms “oligomer” and “oligomeric compound” include oligonucleotides, oligonucleotide analogs, oligonucleosides and chimeric oligomeric compounds where there are more than one type of internucleoside linkages dividing the oligomeric compound into regions. Whereas the term “oligonucleotide” has a well defined meaning in the art, the term “oligomeric compound” or “oligomer” is intended to be broader, inclusive of oligomers having all manner of modifications known in the art.

Heterocyclic base moieties (often referred to in the art simply as “bases”) amenable to the present invention includes both naturally and non-naturally occurring nucleobases. Heterocyclic base moieties further may be protected wherein one or more functionalities of the base bears a protecting group. As used herein, the terms “unmodified nucleobase” or “natural nucleobase” include the purine bases adenine and guanine, and the pyrimidine bases thymine, cytosine and uracil. Additional unmodified or natural nucleobases are known in the art. Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993.

Certain nucleobases are particularly useful for increasing the binding affinity of the oligomeric compounds and hence are preferred in certain embodiments of the present invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Id., pages 276-278) and are presently preferred base substitutions, even more particularly when combined with 2′-methoxyethyl sugar modifications.

Representative United States patents that teach the preparation of modified nucleobases include, but are not limited to, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, certain of which are commonly owned, and each of which is herein incorporated by reference, and commonly owned United States patent application Ser. No. 08/762,488, filed on Dec. 10, 1996, also herein incorporated by reference.

The preferred sugar moieties are deoxyribose or ribose. However, other sugar substitutes known in the art are also amenable to the present invention. One such substitute sugar has the ring O replaced with another moiety. Representative substitutions for ring O include, but are not limited to, S, CH₂, CBF, and CF₂. See, e.g., Secrist et al., Abstract 21, Program & Abstracts, Tenth International Roundtable, Nucleosides, Nucleotides and their Biological Applications, Park City, Utah, Sep. 16-20, 1992, hereby incorporated by reference in its entirety.

A further preferred substitute sugar has been termed a locked nucleic acid (LNA) in which a 2′-C, 4′-C-oxymethylene linkage on the sugar locks the sugar into a particular conformation. The linkage is preferably a methelyne (—CH₂—)ₙ group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tₘ=+3 to +10 C), stability towards 3′-exonucleolytic degradation and good solubility properties.

Novel types of LNA-modified oligonucleotides, as well as the LNAs, are useful in a wide range of diagnostic and therapeutic applications. Among these are antisense applications, PCR applications, strand-displacement oligomers, substrates for nucleic acid polymerases and generally as nucleotide based drugs.

Potent and nontoxic antisense oligonucleotides containing LNAs have been described (Wahlestedt et al., Proc. Natl. Acad. Sci. U.S.A., 2000, 97, 5633-5638.) The authors have demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

The first analogs of LNA, phosphorothioate-LNA and 2′-thio-LNAs, have been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., PCT International Application WO 98-DK393 19980914). Furthermore, synthesis of 2′-amino-LNA, a novel conformationally restricted high-affinity oligonucleotide analog with a handle has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

As used herein, the term “sugar substituent group” refers to groups that are attached to sugar moieties of nucleosides that comprise compounds or oligomers of the invention. Sugar substituent groups are covalently attached at sugar 2′, 3′ and 5′-positions. In some preferred embodiments, the sugar substituent group has an oxygen atom bound directly to the 2′, 3′ and/or 5′-carbon atom of the sugar. Preferably, sugar substituent groups are attached at 2′-positions although sugar substituent groups may also be located at 3′ and 5′ positions.

Sugar substituent groups amenable to the present invention include fluoro, O-alkyl, O-alkylamino, O-alkylalkoxy, protected O-alkylamino, O-alkylaminoalkyl, O-alkyl imidazole, and polyethers of the formula (O-alkyl)ₘ, where m is 1 to about 10. Preferred among these polyethers are linear and cyclic polyethylene glycols (PEGs), and (PEG)-containing groups, such as crown ethers and those which are disclosed by Ouchi et al. (Drug Design and Discovery 1992, 9, 93), Ravasio et al. (J. Org. Chem. 1991, 56, 4329) and Delgardo et. al. (Critical Reviews in Therapeutic Drug Carrier Systems 1992, 9, 249), each of which is herein incorporated by reference in its entirety. Further sugar modifications are disclosed in Cook, P. D., Anti-Cancer Drug Design, 1991, 6, 585-607. Fluoro, O-alkyl, O-alkylamino, O-alkyl imidazole, O-alkylaminoalkyl, and alkyl amino substitution is described in U.S. patent application Ser. No. 08/398,901, filed Mar. 6, 1995, entitled Oligomeric Compounds having Pyrimidine Nucleotide(s) with 2′ and 5′ Substitutions, hereby incorporated by reference in its entirety.

Additional sugar substituent groups amenable to the present invention include —SR and —NR₂ groups, wherein each R is, independently, hydrogen, a protecting group or substituted or unsubstituted alkyl, alkenyl, or alkynyl. 2′-SR nucleosides are disclosed in U.S. Pat. No. 5,670,633, issued Sep. 23, 1997, hereby incorporated by reference in its entirety. The incorporation of 2′-SR monomer synthons are disclosed by Hamm et al., J. Org. Chem., 1997, 62, 3415-3420. 2′-NR₂ nucleosides are disclosed by Goettingen, M., J. Org. Chem., 1996, 61, 6273-6281; and Polushin et al., Tetrahedron Lett., 1996, 37, 3227-3230.

Further representative sugar substituent groups amenable to the present invention include those having one of formula I or II: 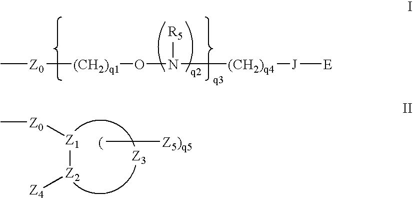
wherein:
Z₀ is O, S or NH;
J is a single bond, O or C(═O);
E is C₁-C₁₀ alkyl, N(R₅)(R₆), N(R₅)(R₇), N═C(R₅ₐ)(R₆ₐ), N═C(R₅ₐ)(R₇ₐ) or has formula IV; 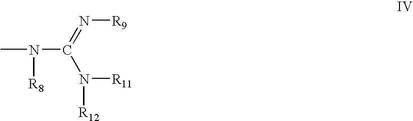
each R₈, R₉, R₁₁ and R₁₂ is, independently, hydrogen, C(O)R₁₃, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, R₁₁ and R₁₂, together form a phthalimido moiety with the nitrogen atom to which they are attached;
each R₁₃ is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
R₅ is T-L,
T is a bond or a linking moiety;
L is a chemical functional group, a conjugate group or a solid support material;
each R₅ and R₆ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl and alkynyl. Further representative alkyl substituents are disclosed in U.S. Pat. No. 5,212,295, at column 12, lines 41-50, hereby incorporated by reference in its entirety.
or R₅ and R₆, together, are a nitrogen protecting group or are joined in a ring structure that optionally includes an additional heteroatom selected from N and O or a chemical functional group;
each R₅ₐ and R₆ₐ is, independently, H, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl and alkynyl. Further representative alkyl substituents are disclosed in U.S. Pat. No. 5,212,295, at column 12, lines 41-50, hereby incorporated by reference in its entirety.
R₇ₐ is -T-L;
each R₁₄ and R₁₅, is, independently, H, C₁-C₁₀ alkyl, a nitrogen protecting group, or R₁₄ and R₁₅, together, are a nitrogen protecting group;
or R₁₄ and R₁₅ are joined in a ring structure that optionally includes an additional heteroatom selected from N and O;
Z₄ is OX, SX, or N(X)₂;
each X is, independently, H, C₁-C₈ alkyl, C₁-C₈ haloalkyl, C(═NH)N(H)R₁₆, C(═O)N(H)R₁₆ or OC(═O)N(H)R₁₆;
R1₁₆ is H or C₁-C₈ alkyl;
Z₁, Z₂ and Z₃ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;
Z₅ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(R₅)(R₆)OR₅, halo, SR₁ or CN;
each q1 is, independently, an integer from 1 to 10;
each q2 is, independently, 0 or 1;
q3 is 0 or an integer from 1 to 10;
q4 is an integer from 1 to 10;
q5 is from 0, 1 or 2; and
   provided that when q3 is 0, q4 is greater than 1.

Representative sugar substituents of formula I are disclosed in U.S. patent application Ser. No. 09/130,973, filed Aug. 7, 1998, now U.S. Pat. No. 6,172,209, entitled “Capped 2′-Oxyethoxy Oligonucleotides,” hereby incorporated by reference in its entirety.

Representative cyclic sugar substituents of formula II are disclosed in U.S. patent application Ser. No. 09/123,108, filed Jul. 27, 1998, entitled “RNA Targeted 2′-Modified Oligonucleotides that are Conformationally Preorganized,” hereby incorporated by reference in its entirety.

Particularly preferred sugar substituent groups include O[(CH₂)ₙO]ₘCH₃, O(CH₂)ₙOCH₃, O(CH₂)ₙNH_{2,} O(CH₂)ₙCH_{3,} O(CH₂)ₙONH₂ and O(CH₂)ₙON[(CH₂)ₙCH₃)]₂ where n and m are from 1 to about 10.

Some preferred oligomeric compounds of the invention contain, in addition to a 2′-O-acetamido modified nucleoside, at least one nucleoside having one of the following at the 2′-position: C₁ to C₁₀ lower alkyl, substituted lower alkyl, alkaryl, aralkyl, O-alkaryl or O-aralkyl, SH, SCH₃, OCN, Cl, Br, CN, CF_{3,} OCF_{3,} SOCH_{3,} SO₂CH_{3,} ONO_{2,} NO_{2,} N_{3,} NH_{2,} heterocycloalkyl, heterocycloalkaryl, aminoalkylamino, polyalkylamino, substituted silyl, an RNA cleaving group, a reporter group, an intercalator, a group for improving the pharmacokinetic properties of an oligomeric compound, or a group for improving the pharmacodynamic properties of an oligomeric compound, and other substituents having similar properties. A preferred modification includes 2′-methoxyethoxy [2′-O—CH₂CH₂OCH₃, also known as 2′-O-(2-methoxyethyl) or 2′-MOE] (Martin et al., Helv. Chim. Acta, 1995, 78, 486), i.e., an alkoxyalkoxy group. A further preferred modification is 2′-dimethylaminooxyethoxy, i.e., a O(CH₂)₂ON(CH₃)₂ group, also known as 2′-DMAOE, as described in co-owned U.S. patent application Ser. No. 09/016,520, filed on Jan. 30, 1998, now U.S. Pat. No. 6,127,533, the contents of which are herein incorporated by reference.

Other preferred modifications include 2′-methoxy (2′-O—CH₃), 2′-aminopropoxy (2′-OCH₂CH₂CH₂NC₂) and 2′-fluoro (2′-F). Similar modifications may also be made at other positions on nucleosides and oligomers, particularly the 3′ position of the sugar on the 3′ terminal nucleoside or in 2′-5′ linked oligomers and the 5′ position of 5′ terminal nucleoside. Oligomers may also have sugar mimetics such as cyclobutyl moieties in place of the pentofuranosyl sugar. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920, certain of which are commonly owned, and each of which is herein incorporated by reference, and commonly owned U.S. patent application Ser. No. 08/468,037, filed on Jun. 5, 1995, also herein incorporated by reference.

Sugars having O-substitutions on the ribosyl ring are also amenable to the present invention. Representative substitutions for ring O include, but are not limited to, S, CH₂, CHF, and CF₂. See, e.g., Secrist et al., Abstract 21, Program & Abstracts, Tenth International Roundtable, Nucleosides, Nucleotides and their Biological Applications, Park City, Utah, Sep. 16-20, 1992, hereby incorporated by reference in its entirety.

Heterocyclic ring structures of the present invention can be fully saturated, partially saturated, unsaturated or with a polycyclic heterocyclic ring each of the rings may be in any of the available states of saturation. Heterocyclic ring structures of the present invention also include heteroaryl which includes fused systems including systems where one or more of the fused rings contain no heteroatoms. Heterocycles, including nitrogen heterocycles, according to the present invention include, but are not limited to, imidazole, pyrrole, pyrazole, indole, 1H-indazole, α-carboline, carbazole, phenothiazine, phenoxazine, tetrazole, triazole, pyrrolidine, piperidine, piperazine and morpholine groups. A more preferred group of nitrogen heterocycles includes imidazole, pyrrole, indole, and carbazole groups.

The present invention provides oligomeric compounds comprising a plurality of linked nucleosides wherein the preferred internucleoside linkage is a 3′,5′-linkage. Alternatively, 2′,5′-linkages can be used (as described in U.S. application Ser. No. 09/115,043, filed Jul. 14, 1998). A 2′,5′-linkage is one that covalently connects the 2′-position of the sugar portion of one nucleotide subunit with the 5′-position of the sugar portion of an adjacent nucleotide subunit.

The oligonucleotides of the present invention are from about 5 to about 50 bases in length. Preferably, the oligonucleotides of the invention are from 8 to about 30 bases, and more preferably from about 15 to about 25 bases in length

In one preferred embodiment of the invention, blocked/protected and appropriately activated nucleosidic monomers are incorporated into oligomeric compounds in the standard manner for incorporation of a normal blocked and activated standard nucleotide. For example, a DMT phosphoramidite nucleosidic monomer is selected that has a 2′-phosphorothioate monoester moiety that can include protection of functional groups. The nucleosidic monomer is added to the growing oligomeric compound by treating with the normal activating agents, as is known is the art, to react the phosphoramidite moiety with the growing oligomeric compound. This may be followed by removal of the DMT group in the standard manner and continuation of elongation of the oligomeric compound with normal nucleotide amidite units. Alternatively, the phosphoramidite can be intended to be the terminus of the oligomeric compound in which case it may be purified with the DMT group on or off following cleavage from the solid support. There are a plurality of alternative methods for preparing oligomeric compounds of the invention that are well known in the art. The phosphoramidite method is meant as illustrative of one of these methods.

In the context of this specification, alkyl (generally C₁-C₁₀), alkenyl (generally C₂-C₁₀), and alkynyl (generally C₂-C₁₀) groups include but are not limited to substituted and unsubstituted straight chain, branch chain, and alicyclic hydrocarbons, including generally C₁-C₂₀ alkyl groups, and also including other higher carbon alkyl groups. Further examples include 2-methylpropyl, 2-methyl-4-ethylbutyl, 2,4-diethylbutyl, 3-propylbutyl, 2,8-dibutyldecyl, 6,6-dimethyloctyl, 6-propyl-6-butyloctyl, 2-methylbutyl, 2-methylpentyl, 3-methylpentyl, 2-ethylhexyl and other branched chain groups, allyl, crotyl, propargyl, 2-pentenyl and other unsaturated groups containing a pi bond, cyclohexane, cyclopentane, adamantane as well as other alicyclic groups, 3-penten-2-one, 3-methyl-2-butanol, 2-cyanooctyl, 3-methoxyheptanal 3-nitrobutyl, 4-isopro-poxydodecyl, 4-azido-2-nitrodecyl, 5-mercaptononyl, 4-amino-1-pentenyl as well as other substituted groups.

Further, in the context of this invention, a straight chain compound means an open chain compound, such as an aliphatic compound, including alkyl, alkenyl, or alkynyl compounds; lower alkyl, alkenyl, or alkynyl as used herein include but are not limited to hydrocarbyl compounds from about 1 to about 6 carbon atoms. A branched compound, as used herein, comprises a straight chain compound, such as an alkyl, alkenyl, alkynyl compound, which has further straight or branched chains attached to the carbon atoms of the straight chain. A cyclic compound, as used herein, refers to closed chain compounds, i.e. a ring of carbon atoms, such as an alicyclic or aromatic compound. The straight, branched, or cyclic compounds may be internally interrupted, as in alkoxy or heterocyclic compounds. In the context of this invention, internally interrupted means that the carbon chains may be interrupted with heteroatoms such as O, N, or S. However, if desired, the carbon chain may have no heteroatoms.

As used herein, “polyamine” refers to a moiety containing a plurality of amine or substituted amine functionalities. Polyamines according to the present invention have at least two amine functionalities. “polypeptide” refers to a polymer comprising a plurality of amino acids linked by peptide linkages, and includes dipeptides and tripeptides. The amino acids may be naturally-occurring or non-naturally-occurring amino acids. Polypeptides according to the present invention comprise at least two amino acids.

As used herein, the term oligonucleoside includes oligomers or polymers containing two or more nucleoside subunits having a non-phosphorous linking moiety. Oligonucleosides according to the invention have monomeric subunits or nucleosides having a ribofuranose moiety attached to a heterocyclic base moiety through a glycosyl bond.

Oligonucleotides and oligonucleosides can be joined to give a chimeric oligomeric compound. Phosphorus and non phosphorus containing linking groups that can be used to prepare oligomeric compounds of the invention are well documented in the prior art and include without limitation the following:

Phosphorus Containing Linkages
phosphorodithioate (—O—P(S)(S)—O—);
phosphorothioate (—O—P(S)(O)—O—);
phosphonate (—O—P(J)(O)—O—);
phosphoramidate (—O—P(O)(NJ)-O—);
phosphorothioamidate (—O—P(O)(J)-S—);
thionoalkylphosphonate (—O—P(S)(J)-O—);
phosphotriesters (—O—P(O J)(O)—O—);
thionoalkylphosphotriester (—O—P(O)(OJ)-S—);
boranophosphate (—R⁵—P(O)(O)-J-);

Non-Phosphorus Containing Linkages
thiodiester (—O—C(O)—S—);
thionocarbamate (—O—C(O)(NJ)-S—);
siloxane (—O—Si(J)₂-O—);
carbamate (—O—C(O)NH— and —NH—C(O)—O—)
sulfamate (—O—S(O)(O)—N— and —N—(O)(O)—N—;
morpholino sulfamide (—O—S(O)(N(morpholino)-);
sulfonamide (—O—SO₂—NH—);
sulfide (—CH₂—S—CH₂—);
sulfonate (—O—SO₂—CH₂—);
N,N′-dimethylhydrazine (—CH₂—N(CH₃)—N(CH₃)—);
thioformacetal (—S—CH₂—O—);
formacetal (—O—CHr-O—);
thioketal (—S—C(J)₂-O—); and
   ketal (—O—C(J)₂-O—);
amine (—NH—CH₂—CH₂—);
hydroxylamine (—CH₂—N(J)-O—);
hydroxylimine (—CH═N—O—); and
hydrazinyl (—CH₂—N(H)—N(H)—).

“J” denotes a substituent group which is commonly hydrogen or an alkyl group, but which can be a more complicated group that varies from one type of linkage to another.

In addition to linking groups as described above that involve the modification or substitution of one or more of the —O—P(O)₂—O— atoms of a naturally occurring linkage, included within the scope of the present invention are linking groups that include modification of the 5′-methylene group as well as one or more of the atoms of the naturally occurring linkage. Linking groups (or linkages) of this type are well documented in the literature and include without limitation the following: amides (—CH₂—CH₂—N(H)—C(O)) and —CH₂—O—N═CH—; and alkylphosphorus (—C(J)₂-P(═O)(OJ)-C(J)₂-C(J)₂-), wherein J is as described above.

Synthetic schemes for the synthesis of the substitute internucleoside linkages described above are disclosed in: WO 91/08213; WO 90/15065; WO 91/15500; WO 92/20822; WO 92/20823; WO 91/15500; WO 89/12060; EP 216860; U.S. 92/04294; U.S. 90/03138; U.S. 91/06855; U.S. 92/03385; U.S. 91/03680; U.S. patent Ser. Nos. 07/990,848; 07,892,902; 07/806,710; 07/763,130; 07/690,786; U.S. Pat. Nos. 5,466,677; 5,034,506; 5,124,047; 5,278,302; 5,321,131; 5,519,126; 4,469,863; 5,455,233; 5,214,134; 5,470,967; 5,434,257; Stirchak, E. P., et al., Nucleic Acid Res., 1989, 17, 6129-6141; Hewitt, J. M., et al., 1992, 11, 1661-1666; Sood, A., et al., J. Am. Chem. Soc., 1990, 112, 9000-9001; Vaseur, J. J. et al., J. Amer. Chem. Soc., 1992, 114, 4006-4007; Musichi, B., et al., J. Org. Chem., 1990, 55, 4231-4233; Reynolds, R. C., et al., J. Org. Chem., 1992, 57, 2983-2985; Mertes, M. P., et al., J. Med. Chem., 1969, 12, 154-157; Mungal, W. S., et al., J. Org. Chem., 1977, 42, 703-706; Stirchak, E. P., et al., J. Org. Chem., 1987, 52, 4202-4206; Coull, J. M., et al., Tet. Lett., 1987, 28, 745; and Wang, H., et al., Tet. Lett., 1991, 32, 7385-7388.

Other modifications can be made to the sugar, to the base, or to the phosphate group of the nucleoside. Representative modifications are disclosed in International Publication Numbers WO 91/10671, published Jul. 25, 1991, WO 92/02258, published Feb. 20, 1992, WO 92/03568, published Mar. 5, 1992, and U.S. Pat. Nos. 5,138,045, 5,218,105, 5,223,618 5,359,044, 5,378,825, 5,386,023, 5,457,191, 5,459,255, 5,489,677, 5,506,351, 5,541,307, 5,543,507, 5,571,902, 5,578,718, 5,587,361, 5,587,469, all assigned to the assignee of this application. The disclosures of each of the above referenced publications are herein incorporated by reference.

The attachment of conjugate groups to oligonucleotides and analogs thereof is well documented in the prior art. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, phospholipids, biotin, phenazine, phenanthridine, antiraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992, U.S. Pat. No. 5,578,718, issued Jul. 1, 1997, and U.S. Pat. No. 5,218,105. Each of the foregoing is commonly assigned with this application. The entire disclosure of each is incorporated herein by reference.

Preferred conjugate groups amenable to the present invention include lipid moieties such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

Other groups for modifying antisense properties include RNA cleaving complexes, pyrenes, metal chelators, porphyrins, alkylators, hybrid intercalator/ligands and photo-crosslinking agents. RNA cleavers include o-phenanthroline/Cu complexes and Ru(bipyridine)₃²⁺ complexes. The Ru(bpy)₃²⁺ complexes interact with nucleic acids and cleave nucleic acids photochemically. Metal chelators include EDTA, DTPA, and o-phenanthroline. Alkylators include compounds such as iodoacetamide. Porphyrins include porphine, its substituted forms, and metal complexes. Pyrenes include pyrene and other pyrene-based carboxylic acids that could be conjugated using the similar protocols.

Hybrid intercalator/ligands include the photonuclease/intercalator ligand 6-[[[9-[[6-(4-nitrobenzamido)hexyl]amino]acridin-4-yl]carbonyl]amino]hexanoyl-penta-fluorophenyl ester. This compound has two noteworthy features: an acridine moiety that is an intercalator and a p-nitro benzamido group that is a photonuclease.

Photo-crosslinking agents include aryl azides such as, for example, N-hydroxysucciniimidyl-4-azidobenzoate (HSAB) and N-succinimidyl-6(-4′-azido-2′-nitrophenyl-amino)hexanoate (SANPAH). Aryl azides conjugated to oligonucleotides effect crosslinking with nucleic acids and proteins upon irradiation, They also crosslink with carrier proteins (such as KLH or BSA), raising antibody against the oligonucleotides.

Vitamins according to the invention generally can be classified as water soluble or lipid soluble. Water soluble vitamins include thiamine, riboflavin, nicotinic acid or niacin, the vitamin B₆ pyridoxal group, pantothenic acid, biotin, folic acid, the B₁₂ cobamide coenzymes, inositol, choline and ascorbic acid. Lipid soluble vitamins include the vitamin A family, vitamin D, the vitamin E tocopherol family and vitamin K (and phytols). The vitamin A family, including retinoic acid and retinol, are absorbed and transported to target tissues through their interaction with specific proteins such as cytosol retinol-binding protein type II (CRBP-II), retinol-binding protein (RBP), and cellular retinol-binding protein (CRBP). These proteins, which have been found in various parts of the human body, have molecular weights of approximately 15 kD. They have specific interactions with compounds of vitamin-A family, especially, retinoic acid and retinol.

In the context of this invention, “hybridization” shall mean hydrogen bonding, which may be Watson-Crick, Hoogsteen or reversed Hoogsteen hydrogen bonding, between complementary nucleotides. For example, adenine and thymine are complementary nucleobases that pair through the formation of hydrogen bonds. “Complementary,” as used herein, also refers to sequence complementarity between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity such that stable and specific binding occurs between the oligonucleotide and the DNA or RNA target. It is understood that an oligonucleotide need not be 100% complementary to its target DNA sequence to be specifically hybridizable. An oligonucleotide is specifically hybridizable when binding of the oligonucleotide to the target DNA or RNA molecule interferes with the normal function of the target DNA or RNA, and there is a sufficient degree of complementarity to avoid non-specific binding of the oligonucleotide to non-target sequences under conditions in which specific binding is desired, i.e. under physiological conditions in the case of in vivo assays or therapeutic treatment, or in the case of in vitro assays, under conditions in which the assays are performed.

Cleavage of oligonucleotides by nucleolytic enzymes requires the formation of an enzyme-substrate complex, or in particular, a nuclease-oligonucleotide complex. The nuclease enzymes will generally require specific binding sites located on the oligonucleotides for appropriate attachment. If the oligonucleotide binding sites are removed or blocked, such that nucleases are unable to attach to the oligonucleotides, the oligonucleotides will be nuclease resist. In the case of restriction endonucleases that cleave sequence-specific palindromic double-stranded DNA, certain binding sites such as the ring nitrogen in the 3- and 7-positions of heterocyclic base moieties have been identified as required binding sites. Removal of one or more of these sites or sterically blocking approach of the nuclease to these particular positions within the oligonucleotide has provided various levels of resistance to specific nucleases.

Compounds of the invention can be utilized as diagnostics, therapeutics and as research reagents and in kits. They can be utilized in pharmaceutical compositions by adding an effective amount of an oligomeric compound of the invention to a suitable pharmaceutically acceptable diluent or carrier. They further can be used for treating organisms having a disease characterized by the undesired production of a protein. The organism can be contacted with an oligomeric compound of the invention having a sequence that is capable of specifically hybridizing with a strand of target nucleic acid that codes for the undesirable protein.

The formulation of therapeutic compositions and their subsequent administration is believed to be within the skill of those in the art. In general, for therapeutics, a patient in need of such therapy is administered an oligomer in accordance with the invention, commonly in a pharmaceutically acceptable carrier, in doses ranging from 0.01 μg to 100 g per kg of body weight depending on the age of the patient and the severity of the disease state being treed. Further, the treatment may be a single dose or may be a regimen that may last for a period of time which will vary depending upon the nature of the particular disease, its severity and the overall condition of the patient, and may extend from once daily to once every 20 years. Following treatment, the patient is monitored for changes in his/her condition and for alleviation of the symptoms of the disease state. The dosage of the oligomer may either be increased in the event the patient does not respond significantly to current dosage levels, or the dose may be decreased if an alleviation of the symptoms of the disease state is observed, or if the disease state has been ablated.

In some cases it may be more effective to treat a patient with an oligomer of the invention in conjunction with other traditional therapeutic modalities. For example, a patient being treated for AIDS may be administered an oligomer in conjunction with AZT, or a patient with atherosclerosis may be treated with an oligomer of the invention following angioplasty to prevent reocclusion of the treated arteries.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligomers, and can generally be estimated based on EC₅₀s found to be effective in in vitro and in vivo animal models. In general, dosage is from 0.01 μg to 100 g per kg of body weight, and may be given once or more daily, weekly, monthly or yearly, or even once every 2 to several years.

Following successful treatment, it may be desirable to have the patient undergo maintenance therapy to prevent the recurrence of the disease state, wherein the oligomer is administered in maintenance doses, ranging from 0.01 μg to 100 g per kg of body weight, once or more daily, to once every several years.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives.

The present invention can be practiced in a variety of organisms ranging from unicellular prokaryotic and eukaryotic organisms to multicellular eukaryotic organisms. Any organism that utilizes DNA-RNA transcription or RNA-protein translation as a fundamental part of its hereditary, metabolic or cellular machinery is susceptible to such therapeutic and/or prophylactic treatment. Seemingly diverse organisms such as bacteria, yeast, protozoa, algae, plant and higher animal forms, including warm-blooded animals, can be treated in this manner. Further, since each of the cells of multicellular eukaryotes also includes both DNA-RNA transcription and RNA-protein translation as an integral part of their cellular activity, such therapeutics and/or diagnostics can also be practiced on such cellular populations. Furthermore, many of the organelles, e.g. mitochondria and chloroplasts, of eukaryotic cells also include transcription and translation mechanisms. As such, single cells, cellular populations or organelles also can be included within the definition of organisms that are capable of being treated with the therapeutic or diagnostic oligonucleotides of the invention. As used herein, therapeutics is meant to include both the eradication of a disease state, killing of an organism, e.g. bacterial, protozoan or other infection, or control of aberrant or undesirable cellular growth or expression.

The current method of choice for the preparation of oligomeric compounds uses support media. Support media is used to attach a first nucleoside or larger nucleosidic synthon which is then iteratively elongated to give a final oligomeric compound. Support media can be selected to be insoluble or have variable solubility in different solvents to allow the growing oligomer to be kept out of or in solution as desired. Traditional solid supports are insoluble and are routinely placed in a reaction vessel while reagents and solvents react and or wash the growing chain until cleavage frees the final oligomer. More recent approaches have introduced soluble supports including soluble polymer supports to allow precipitating and dissolving the bound oligomer at desired points in the synthesis (Gravert et al., Chem. Rev., 1997, 97, 489-510). Representative support media that are amenable to the methods of the present invention include without limitation: controlled pore glass (CPG); oxalyl-controlled pore glass (see, e.g., Alul, et al., Nucleic Acids Research 1991, 19, 1527); TENTAGEL Support, (see, e.g., Wright, et al., Tetrahedron Letters 1993, 34, 3373); or POROS, a copolymer of polystyrene/divinylbenzene available from Perceptive Biosystems. The use of a soluble support media, poly(ethylene glycol), with molecular weights between 5 and 20 kDa, for large-scale synthesis of phosphorothioate oligonucleotides is described in, Bonora et al., Organic Process Research & Development, 2000, 4, 225-231.

Equipment for support synthesis of oligomeric compounds is sold by several vendors including, for example, Applied Biosystems (Foster City, Calif.). Any other means for such synthesis known in the art may additionally or alternatively be employed. Suitable solid phase techniques, including automated synthesis techniques, are described in F. Eckstein (ed.), Oligonucleotides and Analogues, a Practical Approach, Oxford University Press, New York (1991).

Solid-phase synthesis relies on sequential addition of nucleotides to one end of a growing oligonucleotide chain. Typically, a first nucleoside (having protecting groups on any exocyclic functional groups such as amines) is attached to an appropriate glass bead support and activated phosphite compounds (typically nucleotide phosphoramidites, also bearing appropriate protecting groups) are added stepwise to elongate the growing oligonucleotide. Additional methods for solid-phase synthesis may be found in Caruthers U.S. Pat. Nos. 4,415,732; 4,458,066; 4,500,707; 4,668,777; 4,973,679; and 5,132,418; and Koster U.S. Pat. No. 4,725,677 and Re. 34,069.

Solid supports according to the invention include controlled pore glass (CPG), oxalyl-controlled pore glass (see, e.g., Alul, et al., Nucleic Acids Research 1991, 19, 1527), TentaGel Support—an aminopolyethyleneglycol derivatized support (see, e.g., Wright, et al., Tetrahedron Letters 1993, 34, 3373) or Poros—a copolymer of polystyrene/divinylbenzene.

Oligonucleotides are synthesized by standard solid phase nucleic acid synthesis using an automated synthesizer such as Model 380B (Perkin Elmer/Applied Biosystems) or MilliGen/Biosearch 7500 or 8800. Triester, phosphoramidite, or hydrogen phosphonate coupling chemistries (Oligonucleotides: Antisense Inhibitors of Gene Expression. M. Caruthers, p. 7, J. S. Cohen (Ed.), CRC Press, Boca Raton, Fla., 1989) are used with these synthesizers to provide the desired oligonucleotides. The Beaucage reagent (J. Amer. Chem. Soc., 1990, 112, 1253) or elemental sulfur (Beaucage et al., Tet. Lett., 1981, 22, 1859) is used with phosphoramidite or hydrogen phosphonate chemistries to provide phosphorothioate oligonucleotides.

Useful sulfurizing agents include Beaucage reagent described in, for example, Iyer et al., J Am Chem Soc, 112, 1253-1254 (1990); and Iyer et al., J Org Chem, 55, 4693-4699 (1990); tetraethyl-thiuram disulfide as described in Vu et al., Tetrahedron Lett, 32, 3005-3007 (1991); dibenzoyl tetrasulfide as described in Rao et al., Tetrahedron Lett, 33, 4839-4842 (1992); di(phenylacetyl)disulfide, as described in Kamer et al., Tetrahedron Lett, 30, 6757-6760 (1989); Bis(O,O-diisopropoxy phosphinothioyl)disulfide, Stec., Tetrahedron Letters, 1993, 34, 5317-5320; sulfur; and sulfur in combination with ligands like triaryl, trialkyl or triaralkyl or trialkaryl phosphines. Useful oxidizing agents, in addition to those set out above, include iodine/tetrahydro furan/water/pyridine; hydrogen peroxide/water; tert-butyl hydroperoxide; or a peracid like m-chloroperbenzoic acid. In the case of sulfurization, the reaction is performed under anhydrous conditions with the exclusion of air, in particular oxygen; whereas, in the case of oxidation the reaction can be performed under aqueous conditions.

The requisite nucleosides (A, G, C, T(U)), and other nucleosides having modified sugar and/or modified bases are prepared, utilizing procedures as described below.

During the synthesis of nucleoside monomers and oligomeric compounds of the invention, chemical protecting groups can be used to facilitate conversion of one or more functional groups while other functional groups are rendered inactive. A number of chemical functional groups can be introduced into compounds of the invention in a blocked form and subsequently deblocked to form a final, desired compound. In general, a blocking group renders a chemical functionality of a molecule inert to specific reaction conditions and can later be removed from such functionality in a molecule without substantially damaging the remainder of the molecule (Green and Wuts, Protective Groups in Organic Synthesis, 2d edition, John Wiley & Sons, New York, 1991). For example, amino groups can be blocked as phthalimido groups, as 9-fluorenylmethoxycarbonyl (FMOC) groups, and with triphenylmethylsulfenyl, t-BOC, benzoyl or benzyl groups. Carboxyl groups can be protected as acetyl groups. Representative hydroxyl protecting groups are described by Beaucage et al., Tetrahedron 1992, 48, 2223. Preferred hydroxyl protecting groups are acid-labile, such as the trityl, monomethoxytrityl, dimethoxytrityl, trimethoxytrityl, 9-phenylxanthine-9-yl (Pixyl) and 9-(p-methoxyphenyl)xanthine-9-yl (MOX) groups. Chemical functional groups can also be “blocked” by including them in a precursor form. Thus, an azido group can be used considered as a “blocked” form of an amine since the azido group is easily converted to the amine. Representative protecting groups utilized in oligonucleotide synthesis are discussed in Agrawal et al., Protocols for Oligonucleotide Conjugates, Eds, Humana Press; New Jersey, 1994; Vol. 26 pp. 1-72.

Among other uses, the oligomeric compounds of the invention are useful in a ras-luciferase fusion system using ras-luciferase transactivation. As described in International Publication Number WO 92/22651, published Dec. 23, 1992 and U.S. Pat. Nos. 5,582,972 and 5,582,986, commonly assigned with this application, the entire contents of which are herein incorporated by reference, the ras oncogenes are members of a gene family that encode related proteins that are localized to the inner face of the plasma membrane. Ras proteins have been shown to be highly conserved at the amino acid level, to bind GTP with high affinity and specificity, and to possess GTPase activity. Although the cellular function of ras gene products is unknown, their biochemical properties, along with their significant sequence homology with a class of signal-transducing proteins known as GTP binding proteins, or G proteins, suggest that ras gene products play a fundamental role in basic cellular regulatory functions relating to the transduction of extracellular signals across plasma membranes.

Three ras genes, designated H-ras, K-ras, and N-ras, have been identified in the mammalian genome. Mammalian ras genes acquire transformation-inducing properties by single point mutations within their coding sequences. Mutations in naturally occurring ras oncogenes have been localized to codons 12, 13 and 61. The most commonly detected activating ras mutation found in human tumors is in codon-12 of the H-ras gene in which a base change from GGC to GTC results in a glycine-to-valine substitution in the GTPase regulatory domain of the ras protein product. This single amino acid change is thought to abolish normal control of ras protein function, thereby converting a normally regulated cell protein to one that is continuously active. It is believed that such deregulation of normal ras protein function is responsible for the transformation firm normal to malignant growth.

In addition to modulation of the ras gene, the oligomeric compounds of the present invention that are specifically hybridizable with other nucleic acids can be used to modulate the expression of such other nucleic acids. Examples include the raf gene, a naturally present cellular gene which occasionally converts to an activated form that has been implicated in abnormal cell proliferation and tumor formation. Other examples include those relating to protein kinase C (PKC) that have been found to modulate the expression of PKC, those related to cell adhesion molecules such as ICAM, those related to multi-drug resistance associated protein, and viral genomic nucleic acids include HIV, herpesviruses, Epstein-Barr virus, cytomegalovirus, papillomavirus, hepatitis C virus and influenza virus (see, U.S. Pat. Nos. 5,166,195, 5,242,906, 5,248,670, 5,442,049,5,457,189,5,510,476,5,510,239,5,514,577, 5,514,786,5,514,788, 5,523,389, 5,530,389,5,563,255,5,576,302,5,576,902,5,576,208, 5,580,767,5,582,972, 5,582,986, 5,591,720, 5,591,600 and 5,591,623, commonly assigned with this application, the disclosures of which are herein incorporated by reference).

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative, not limiting.

### EXAMPLES

### General

Phosphoramidites (including 5′-DMT-thymidine-3′-O-(2-cyanoethyl)-N,N-diisopropylphosphoramidite; 5′-DMT-N²-isobutyryl-2′-deoxyguanosine-3′-O-(2-cyanoethyl)-N,N-diisopropylphosphoramidite; 5′-DMT-N⁴-benzoyl-2′-deoxycytidine-3′-O-(2-cyanoethyl)-N,N-diisopropylphosphoramidite; and 5′-DMT-N⁶-benzoyl-2′-deoxy-adenosine-3′-O-(2-cyanoethyl)-N,N-diisopropylphosphoramidite) and other reagents used in the automated synthesis of oligonucleotides were purchased from commercial sources (Glen Research, Sterling, Va.; Amersham Pharmacia Biotech Inc., Piscataway, N.J.; Cruachem Inc., Aston, Pa.; Chemgenes Corporation, Waltham, Mass.; Proligo LLC, Boulder, Colo.; PE Biosystems, Foster City Calif.; Beckman Coulter Inc., Fullerton, Calif.).

### Example 1

### General Procedure for the Preparation of an Oligomeric Compound Having a Phosphorothioate Monoester at the 3′-terminus (Preparation of Deoxyphosphorothioate: SEQ ID NO:1, GCCCAAGCTG GCATCCGTCA, ISIS # 2302)

5′-O-DMT-thymidine derivatized Primer HL 30 support (1.80 g) was packed into a steel reactor vessel (6.3 mL). The DMT group was removed by treatment with a solution of dichloroacetic acid in toluene (3% v/v). The deprotected support-bound nucleoside was washed with acetonitrile then a solution of Phosphate-O™ (5′-Phosphate-ON Reagent, DMTO-CH₂—CH₂—SO₂—CH₂—CH₂—O—P(CN—CH₂—CH₂—O—)—N[CH(CH₃)₂)₂, commercially available from Chemgenes Corporation Waltham, Mass.) in acetonitrile (0.2 M) and a solution of 1-H-tetrazole in acetonitrile (0.45 M) was added. The mixture was allowed to react for 5 minutes and the solid support was washed with acetonitrile. A solution of phenylacetyl disulfide in 3-picoline-acetonitrile (0.2 M, 1:1, v/v) was added and allowed to react at room temperature for 2 minutes. The product was washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture was removed by washing the product with acetonitrile.

A solution of dichloroacetic acid in toluene (3%, v/v) was added to deprotect the protected hydroxy group and the product was washed with acetonitrile. A solution of 5′-DMT-N⁶-benzoyl-2′-deoxyadenosine-3′-O-(2-cyanoethyl)-N,N-diisopropylphosphoramidite (0.2 M) and a solution of 1-H-tetrazole in acetonitrile (0.45 M) were added and allowed to react for 10 minutes at room temperature. A solution of phenylacetyl disulfide in 3-picoline-acetonitrile (0.2 M, 1:1, v/v) was added and allowed to react at room temperature for 2 minutes. The product was washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture was removed by washing the product with acetonitrile.

A solution of dichloroacetic acid in toluene (3% v/v) was added to deprotect the 5′-hydroxy group and the product washed with acetonitrile. A solution of 5′-DMT-N⁴-benzoyl-2′-deoxycytidine-3′-O-(2-cyanoethyl)-N,N-diisopropylphosphoramidite (0.2 M) and a solution of 1-H-tetrazole in acetonitrile (0.45 M) were added and allowed to react for 5 minutes at room temperature. A solution of phenylacetyl disulfide in 3-picoline-acetonitrile (0.2 M, 1:1, v/v) was added and allowed to react at room temperature for 2 minutes. The product was washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture was removed by washing the product with acetonitrile.

The process of deprotecting the 5′-hydroxyl group, adding a phosphoramidite and an activating agent, sulfurizing and capping with intervening wash cycles was iteratively repeated eighteen additional cycles to prepare the 20 mer (SEQ ID NO: 1) shown above.

The resulting support bound oligonucleotide was treated with aqueous ammonium hydroxide (30%) for 24 h at 60° C. and the products were filtered. The filtrate was concentrated under reduced pressure and a solution of the residue in water was purified by reversed phase HPLC. The appropriate fractions were collected, combined and concentrated in vacuo. A solution of the residue in water was treated with aqueous sodium acetate solution (pH 3.5) for 45 minutes. The title deoxyphosphorothioate 20 mer oligonucleotide having a 3′-terminal phosphorothioate monoester was collected after precipitation by addition of ethanol.

### Example 2

### General Procedure for the Preparation of an Oligomeric Compound Having a Phosphorothioate Monoester at the 5′-terminus (Preparation of Deoxyphosphorothioate: SEQ ID NO:1)

5′-DMT-N⁶-benzoyl-2′-deoxyadenosine derivatized Primer HL 30 support (1.80 g) is packed into a steel reactor vessel (6.3 mL). The DMT group is removed by treatment with a solution of dichloroacetic acid in toluene (3%, v/v). A solution of 5′-DMT-N⁴-benzoyl-2′-deoxycytidine-3′-O-(2-cyanoethyl)-N,N-diisoproylphosphoramidite in acetonitrile (0.2 M) and a solution of 1-H-tetrazole in acetonitrile (0.45 M) are added and allowed to react for 5 minutes at room temperature. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

The process of deprotecting the 5′-hydroxyl group, adding a phosphoramidite and an activating agent, sulfurizing and capping with intervening wash cycles is iteratively repeated eighteen additional cycles to prepare the 20 mer (SEQ ID NO: 1) shown above.

A 3% v/v solution of dichloroacetic acid in toluene is added to deprotect the 5′-hydroxy group and the solid support bound 20 mer is washed with acetonitrile. To the deblocked 20 mer is added a solution of Phosphate-OnJ in acetonitrile (0.2 M) and a solution of 1-H-tetrazole in acetonitrile (0.45 M). The mixture is allowed to react for 5 minutes at room temperature and the product is washed with acetonitrile. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

The support bound oligonucleotide is treated with 30% aqueous ammonium hydroxide for 24 hours at 60° C. and filtered. The filtrate is concentrated under reduced pressure and a solution of the residue in water is purified by reversed phase HPLC. The appropriate fractions are collected, combined and concentrated in vacuo. The residue is dissolved in water and the title deoxyphosphorothioate 20 mer oligonucleotide having a 5′-terminal phosphorothioate monoester is collected after precipitation by addition of ethanol.

### Example 3

### General Procedure for the Preparation of an Oligomeric Compound Having a 2′-phosphorothioate Monoester at the 3′-terminus (Preparation of Deoxyphosphorothioate: SEQ ID NO:1)

5′-O-DMT-thymidine derivatized Primer HL 30 support (1.80 g) is packed into a steel reactor vessel (6.3 mL). The DMT group is removed by treatment with a solution of dichloroacetic acid in toluene (3% v/v). The deprotected support-bound nucleoside is washed with acetonitrile then a solution of Phosphate-O™ in acetonitrile (0.2 M) and a solution of 1-H-tetrazole in acetonitrile (0.45 M) is added. The mixture is allowed to react for 5 minutes at room temperature and the product is washed with aceto-nitrile. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

A solution of dichloroacetic acid in toluene (3% v/v) is added to deprotect the protected hydroxyl group and the product washed with acetonitrile. A solution of 5′-DMT-N⁶-benzoyl-3′-deoxyadenosine-2′-O-(2-cyanoethyl)-N,N-diisopropylphosphoramidite (0.2 M) and a solution of 1-H-tetrazole in acetonitrile (0.45 M) are added and allowed to react for 5 minutes at room temperature. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

A 3% v/v solution of dichloroacetic acid in toluene is added to deprotect the 5′-hydroxy group and the product washed with acetonitrile. A solution of 5′-DMT-N⁴-benzoyl-2′-deoxycytidine-3′-O-(2-cyanoethyl)-N,N-diisopropylphosphoramidite (0.2 M) and a solution of 1-H-tetrazole (0.45 M) in acetonitrile are added and allowed to react for 5 minutes at room temperature. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

The process of deprotecting the 5′-hydroxyl group, adding a phosphoramidite and an activating agent, sulfurizing and capping with intervening wash cycles is iteratively repeated eighteen additional cycles to prepare the 20 mer (SEQ ID NO: 1) shown above.

The support bound oligonucleotide is treated with 30% aqueous ammonium hydroxide for 24 hours at 60° C. and the products filtered. The filtrate is concentrated under reduced pressure and a solution of the residue in water purified by reversed phase HPLC. The appropriate fractions are collected, combined and concentrated in vacuo. The residue is dissolved in water and treated with aqueous sodium acetate solution (pH 3.5) for 45 minutes. The title deoxyphosphorothioate 20 mer oligonucleotide having a 2′-phosphorothioate monoester at the 3′-terminal nucleoside is collected after precipitation by addition of aqueous sodium acetate and ethanol.

### Example 4

### General Procedure for the Preparation of an Oligomeric Compound Having a N⁶-phosphorothioate Monoester at the 3′-terminal Deoxy Adenosine (Preparation of deoxyphosphorothioate: SEQ ID NO:1)

A solution of 5′-O-DMT-2′-deoxyadeonsine (5 mmol) in pyridine is treated with trimethylsilyl chloride (40 mmol). After 30 minutes at room temperature bis (2-cyanoethoxy)-(N,N-diisopropylamino)phosphine (7.5 mmol) is added and the mixture is stirred for 2 hours at room temperature. Diethyldithiocarbonate disulfide (50 mmol) is added and the products stirred at room temperature for 1 hour. The mixture is diluted with dichloromethane, washed with a solution of aqueous sodium hydrogen carbonate, dried over sodium sulfate and concentrated under reduced pressure. The residue is purified by chromatography on silica gel and the appropriate fractions collected, combined and evaporated.

The residue is redissolved in pyridine and succinic anhydride (10 mmol) and 4,4-dimethylaminopyridine (1 mmol) is added. The products are allowed to stir at room temperature overnight then water is added. After a further 10 minutes the mixture is concentrated under reduced pressure. A solution of the residue in dichloromethane is washed with aqueous sodium hydrogen carbonate solution then dried over sodium sulfate and concentrated under reduced pressure. The residue is purified by chromatography on silica gel and the appropriate fractions collected, combined and evaporated.

The above fully protected succinate (1 mmol), dicyclohexylcarbodiimide (4 mmol), 4,4-dimethylaminopyridine (1 mmol) and amino-derivatized Primer HL-30 support (10 g) are shaken together in pyridine for 16 hours at room temperature. The support is collected by filtration and washed with pyridine, methanol and diethyl ether. The dried support is resuspended in a 1:1 v/v mixture of acetic anhydride in acetonitrile (1:4 v/v) and N-methylimidazole-pyridine-acetonitrile (2:3:5 v/v/v) and the products shaken at room temperature for 2 hours. The support is collected by filtration and washed with pyridine, methanol and diethyl ether.

The above derivatized Primer HL 30 support (1.80 g) is packed into a steel reactor vessel (6.3 mL). The DMT group is removed by treatment with a solution of dichloroacetic acid in toluene (3%, v/v). A solution of 5′-DMT-N⁴-benzoyl-2′-deoxy-cytidine-3′-O-(2-cyanoethyl)-N,N-diisopropylphosphoramidite (0.2 M) and a solution of 1-H-tetrazole in acetonitrile (0.45 M) are added and allowed to react for 5 minutes at room temperature. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

The process of deprotecting the 5′-hydroxyl group, adding a phosphoramidite and an activating agent, sulfurizing and capping with intervening wash cycles is iteratively repeated eighteen additional cycles to prepare the 20 mer (SEQ ID NO: 1) shown above.

The support bound oligonucleotide is treated with 30% aqueous ammonium hydroxide for 14 hours at 60° C. and the products filtered. The filtrate is concentrated under reduced pressure and a solution of the residue in water purified by reversed phase HPLC. The appropriate fractions are collected, combined and concentrated in vacuo. A solution of the residue in water is treated with aqueous sodium acetate solution (pH 3.5) for 45 minutes. The title deoxyphosphorothioate 20 mer oligonucleotide having a phosphorothioate monoester covalently attached to the N⁶-position of the 3′-terminal adenosine nucleoside is collected after precipitation by addition of ethanol.

### Example 5

### General Procedure for the Preparation of an Oligomeric Compound Having a N²-phosphorothioate Monoester at the 5′-terminal Deoxy Guanosine (Preparation of Deoxyphosphorothioate: SEQ ID NO:1)

A solution of 5′-O-DMT-2′-deoxyguanosine (5 mmol) in pyridine is treated trimethylsilyl chloride (40 mmol). After 30 minutes at room temperature bis (2-cyanoethoxy)-(N,N-diisopropylamino)phosphine (7.5 mmol) is added and the mixture is stirred for 2 hours at room temperature. Diethyldithiocarbonate disulfide (50 mmol) is added and the products are stirred at room temperature for 1 hour. The mixture is diluted with dichloromethane, washed with a solution of aqueous sodium hydrogen carbonate, dried over sodium sulfate and concentrated under reduced pressure. The residue is purified by chromatography on silica gel and the appropriate fractions collected, combined and concentrated under reduced pressure.

The residue obtained is dissolved in acetonitrile and 2-cyanoethyl-N,N,N′,N′-tetraisopropylphosphorodiamidite (10 mmol) and 1-H-tetrazole (9 mmol) are added. After 2 hours the mixture is diluted with dichloromethane and washed with a solution of aqueous sodium hydrogen carbonate. The organic layer is dried over sodium sulfate and concentrated under reduced pressure. The residue is purified by chromatography on silica gel. The appropriate fractions are collected, pooled and concentrated in vacuo to give 5′-O-DMT-N²-bis (2-cyanoethyl)-thiophosphoroamido-2′-deoxyguanosine-3′-O-(2-cyanoethyl)-N,N-diisopropylphosphoramidite.

5′-O-DMT-N⁶-benzoyl-2′-deoxyadenosine derivatized Primer HL 30 support (1.80 g) is packed into a steel reactor vessel (6.3 mL). The DMT group is removed by treatment with a solution of dichloroacetic acid in toluene (3%, v/v). A solution of 5′-DMT-N⁴-benzoyl-2′-deoxycytidine-3′-O-(2-cyanoethyl)-N,N-diisopropyl-phosphoramidite (0.2 M) and a solution of 1-H-tetrazole in acetonitrile (0.45 M) are added and allowed to react for 5 minutes at room temperature. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

The process of deprotecting the 5′-hydroxyl group, adding a phosphoramidite and an activating agent, sulfurizing and capping with intervening wash cycles is iteratively repeated 17 additional cycles to prepare the 19 mer.

A 3% v/v solution of dichloroacetic acid in toluene is added to deprotect the 5′-hydroxy group and the product washed with acetonitrile. A O₂ M solution of 5′-O-DMT-N²-bis(2-cyanoethyl)-thiophosphoroamido-2′-deoxyguanosine-3′-O-(2-cyanoethyl)-N,N-diisopropylphosphoramidite and a 0.45 M solution of 1-H-tetrazole in acetonitrile are added and allowed to react for 5 minutes at room temperature. A 0.2 M solution of phenylacetyl disulfide in 3-picoline-acetonitrile (1:1 v/v) is added and allowed to react at room temperature for 2 minutes. The product is washed with acetonitrile and a 1:1 v/v mixture of acetic anhydride in acetonitrile (1:4 v/v) and N-methylimidazole-pyridine-acetonitrile (2:3:5 v/v/v) is added. After 2 minutes the capping mixture is removed by washing the product with acetonitrile.

The support bound oligonucleotide is treated with 30% aqueous ammonium hydroxide for 24 hours at 60° C. and the products are filtered. The filtrate is concentrated under reduced pressure and a solution of the residue in water purified by reversed phase HPLC. The appropriate fractions are collected, combined and concentrated in vacuo. A solution of the residue in water is treated with aqueous sodium acetate solution (pH 3.5) for 45 minutes. The title 20 mer having a phosphorothioate monoester covalently attached to the N²-position of the 5′-terminal-2′-deoxyguanosine is isolated after ethanol precipitation.

### Example 6

### General Procedure for the Preparation of an Oligomeric Compound Having an N⁴-phosphorothioate Monoester Attached to an Internal Deoxycytidine (Preparation of Deoxyphosphorothioate: SEQ ID NO:1)

A solution of 5′-O-DMT-2′-deoxycytidine (5 mmol) in pyridine is treated trimethylsilyl chloride (40 mmol). After 30 minutes at room temperature bis (2-cyanoethoxy)-(N,N-diisopropylamino)phosphine (7.5 mmol) is added and the mixture stirred for 2 hours at room temperature. Diethyldithiocarbonate disulfide (50 mmol) is added and the products are stirred at room temperature for 1 hour. The mixture is diluted with dichloromethane, washed with a solution of aqueous sodium hydrogen carbonate, dried over sodium sulfate and concentrated under reduced pressure. The residue is purified by chromatography on silica gel and the appropriate fractions collected, combined and concentrated under reduced pressure.

The residue obtained is dissolved in acetonitrile and 2-cyanoethyl-N,N,N′,N′-tetraisopropylphosphorodiamidite (10 mmol) and 1-H-tetrazole (9 mmol) are added. After 2 hours the mixture is diluted with dichloromethane and washed with a solution of aqueous sodium hydrogen carbonate. The organic layer is dried over sodium sulfate and concentrated under reduced pressure. The residue is purified by chromatography on silica gel. The appropriate fractions are collected, pooled and concentrated in vacuo to give 5′-O-DMT-N⁴-bis (2-cyanoethyl)-thiophosphoroamido-2′-deoxycytidine-3′-O-(2-cyanoethyl)-N,N-diisopropylphosphoramidite.

5′-O-DMT-N⁶-benzoyl-2′-deoxyadenosine derivatized Primer HL 30 support (1.80 g) is packed into a steel reactor vessel (6.3 mL). The DMT group is removed by treatment with a solution of dichloroacetic acid in toluene (3%, v/v). A solution of 5′-DMT-N⁴-benzoyl-2′-deoxycytidine-3′-O-(2-cyanoethyl)-N,N-diisopropyl-phosphoramidite (0.2 M) and a solution of 1-H-tetrazole in acetonitrile (0.45 M) are added and allowed to react for 5 minutes at room temperature. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

The process of deprotecting the 5′-hydroxyl group, adding a phosphoramidite and an activating agent, sulfurizing and capping with intervening wash cycles is iteratively repeated ten additional cycles to prepare the 12 mer.

A 3% v/v solution of dichloroacetic acid in toluene is added to deprotect the 5′-hydroxy group and the product washed with acetonitrile. A 0.2M solution of 5′-O-DMT-N⁴-bis (2-cyanoethyl)-thiophosphoroamido-2′-deoxycytidine-3′-O-(2-cyanoethyl)-N,N-diisopropylphosphoramidite and a 0.45 M solution of 1-H-tetrazole in acetonitrile are added and allowed to react for 5 minutes at room temperature. A 0.2 M solution of phenylacetyl disulfide in 3-picoline-acetonitrile (1:1 v/v) is added and allowed to react at room temperature for 2 minutes. The product is washed with acetonitrile and a 1:1 v/v mixture of acetic anhydride in acetonitrile (1:4 v/v) and N-methylimidazole-pyridine-acetonitrile (2:3:5 v/v/v) is added. After 2 minutes the capping mixture is removed by washing the product with acetonitrile (thereby putting the modified nucleoside at position 13 from the 3′-end).

The process of deprotecting the 5′-hydroxyl group, adding a phosphoramidite and an activating agent, sulfurizing and capping with intervening wash cycles is iteratively repeated 7 additional cycles to prepare the 20-mer.

The support bound oligonucleotide is treated with 30% aqueous ammonium hydroxide for 24 hours at 60° C. and the products are filtered. The filtrate is concentrated under reduced pressure and a solution of the residue in water purified by reversed phase HPLC. The appropriate fractions are collected, combined and concentrated in vacuo. A solution of the residue in water is treated with a solution of aqueous sodium acetate (pH 3.5) for 45 minutes. The title 20 mer having a phosphorothioate monoester attached to the N⁴-position of an internal deoxycytidine is isolated following ethanol precipitation.

### Example 7

### General Procedure for the Preparation of an Oligomeric Compound Having a Phosphorothioate Monoester Attached to the 2′-Position of an Internal Adenosine (Preparation of Deoxyphosphorothioate: SEQ ID NO:1, GCCCAAGCTG GCA*TCCGTCA, A* is Modified Position)

A solution of N⁶-benzoyladenosine (5 mmol) in dimethylformamide is treated with silver nitrate (5 mmol) and di-tert-butylsilyl bis(trifluoromethanesulfonate) (5.5 mmol). After 30 minutes the solvent is removed and a solution of the residue in dichloromethane is washed with aqueous sodium hydrogen carbonate. The organic layer is dried and evaporated in vacuo. To a solution of the residue in acetonitrile is added bis(2-cyano-1,1-dimethylethyl)-N,N-diethylphosphoramidite (5 mmol) and 1-H-tetrazole. After 2 hours diethyldithiocarbonate disulfide is added and the products stirred for a further 1 hour. The solvent is removed under vacuum and a solution of the residue in dichloromethane washed with an aqueous sodium hydrogen carbonate solution. The organic layer is dried over sodium sulfate and concentrated under reduced pressure. The residue is dissolved in tetrahydrofuran and a mixture of HF-pyridine and pyridine added. After 10 minutes the products are poured into an aqueous sodium hydrogen carbonate solution and extracted into dichloromethane. The solution is dried over sodium sulfate and concentrated under reduced pressure. The residue is purified by chromatography and the appropriate fractions combined and concentrated under vacuum.

The resulting 2′-phosphorylated nucleoside (2 mmol) is dissolved in pyridine and dimethoxytrityl chloride (2.2 mmol) added. After 2 hours the solvent is removed and a solution of the residue in dichloromethane washed with an aqueous sodium hydrogen carbonate solution. The organic layer is dried over sodium sulfate and concentrated under reduced pressure.

The residue obtained is dissolved in acetonitrile and 2-cyanoethyl-N,N,N′,N′-tetraisopropylphosphorodiamidite (4 mmol) and 1-H-tetrazole (6 mmol) are added. After 2 hours the mixture is diluted with dichloromethane and washed with a solution of aqueous sodium hydrogen carbonate. The organic layer is dried over sodium sulfate and concentrated under reduced pressure. The residue is purified by chromatography on silica gel. The appropriate fractions are collected, pooled and concentrated in vacua to give 5′-O-DMT-2′-O-(2-cyano-1,1-dimethylethyl)-N⁶-benzoyladenosine-thiophosphate-3′-O-(2-cyanoethyl)-N,N-diisopropylphosphoramidite.

5′-O-DMT-N⁶-benzoyl-2′-deoxyadenosine derivatized Primer HL 30 support (1.80 g) is packed into a steel reactor vessel (6.3 mL). The DMT group is removed by treatment with a solution of dichloroacetic acid in toluene (3%, v/v). A solution of 5′-DMT-N⁴-benzoyl-2′-deoxycytidine-3′-O-(2-cyanoethyl)-N,N-diisopropyl-phosphoramidite (0.2 M) and a solution of 1-H-tetrazole in acetonitrile (0.45 M) are added and allowed to react for 5 minutes at room temperature. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

The process of deprotecting the 5′-hydroxyl group, adding a phosphoramidite and an activating agent, sulfurizing and capping with intervening wash cycles is iteratively repeated 5 additional cycles to prepare a 7-mer.

A 3% v/v solution of dichloroacetic acid in toluene is added to deprotect the 5′-hydroxy group and the product washed with acetonitrile. A 0.2 M solution of 5′-O-DMT-2′-O-(2-cyano-1,1-dimethylethyl)-N⁶-benzoyladenosine-thiophosphate-3′-O-(2-cyanoethyl)-N,N-diisopropylphosphoramidite and a 0.45 M solution of 1-H-tetrazole in acetonitrile are added and allowed to react for 5 minutes at room temperature. A 0.2 M solution of phenylacetyl disulfide in 3-picoline-acetonitrile (1:1 v/v) is added and allowed to react at room temperature for 2 minutes. The product is washed with acetonitrile and a 1:1 v/v mixture of acetic anhydride in acetonitrile (1:4 v/v) and N-methylimidazole-pyridine-acetonitrile (2:3:5 v/v/v) is added. After 2 minutes the capping mixture is removed by washing the product with acetonitrile.

The process of deprotecting the 5′-hydroxyl group, adding a phosphoramidite and an activating agent, sulfurizing and capping with intervening wash cycles is iteratively repeated 12 additional cycles to prepare the 20-mer.

The support bound oligonucleotide is treated with 30% aqueous ammonium hydroxide for 24 hours at 60° C. and the products are filtered. The filtrate is concentrated under reduced pressure and a solution of the residue in water purified by reversed phase HPLC. The appropriate fractions are collected, combined and concentrated in vacuo. A solution of the residue in water is treated with aqueous sodium acetate solution (pH 3.5) for 45 minutes. The title 20 mer having a phosphorothioate monoester attached to the 2′-position of an internally situated uridine residue is isolated following ethanol precipitation.

### Example 8

### General Procedure for the Preparation of an Oligomeric Compound Having a Phosphorothioate Monoester Attached to the 3′-Position of a 3′-Terminal Adenosine (Preparation of Deoxyphosphorothioate: SEQ ID NO:1)

5′-O-DMT-thymidine derivatized Primer HL 30 support (1.80 g) is packed into a steel reactor vessel (6.3 mL). The DMT group is removed by treatment with a solution of dichloroacetic acid in toluene (3% v/v). The deprotected support-bound nucleoside is washed with acetonitrile then a solution of Phosphate-O™ (5′-Phosphate-ON Reagent, DMTO-CH₂—CH₂—SO₂—CH₂—CH₂—O—P(CN—CH₂—CH₂—O—)—N[CH(CH₃)₂]₂, commercially available from Chemgenes Corporation Waltham, Mass.) in acetonitrile (0.2 M) and a solution of 1-H-tetrarole in acetonitrile (0.45 M) is added. The mixture is allowed to react for 5 minutes and the solid support is washed with acetonitrile. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

A solution of dichloroacetic acid in toluene (3%, v/v) is added to deprotect the protected hydroxy group and the product is washed with acetonitrile. A solution of 5′-DMT-N⁶-benzoyl-2′-O-t-butyldimethylsilyladenosine-3′-O-(2-cyanoethyl)-N,N-diisopropylphosphoramidite (0.2 M) and a solution of 1-H-tetrazole in acetonitrile (0.45 M) are added and allowed to react for 10 minutes at room temperature. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

A solution of dichloroacetic acid in toluene (3% v/v) is added to deprotect the 5′-hydroxy group and the product washed with acetonitrile. A solution of 5′-DMT-N⁴-benzoyl-2′-deoxycytidine-3′-O-(2-cyanoethyl)-N,N-diisopropylphosphoramidite (0.2 M) and a solution of 1-H-tetrazole in acetonitrile (0.45 M) are added and allowed to react for 5 minutes at room temperature. A solution of phenylacetyl disulfide in 3-picoline-acetonitrile (0.2 M, 1:1, v/v) is added and allowed to react at room temperature for 2 minutes. The product is washed with acetonitrile followed by a capping mixture (1:1, v/v) of acetic anhydride in acetonitrile (1:4 v/v) and N-methylimidazole-pyridine-acetonitrile (2:3:5, v/v/v). After 2 minutes the capping mixture is removed by washing the product with acetonitrile.

The process of deprotecting the 5′-hydroxyl group, adding a phosphoramidite and an activating agent, sulfurizing and capping with intervening wash cycles is iteratively repeated eighteen additional cycles to prepare the 20 mer.

The resulting support bound oligonucleotide is treated with aqueous ammonium hydroxide (30%) for 24 hours at 60° C. and the products are filtered. The residue is treated with 1M t-butylammonium fluoride in THF for 24 hours at room temperature. The products are concentrated and a solution of the residue in water is purified by reversed phase HPLC. The appropriate fractions are collected, combined and concentrated in vacuo. A solution of the residue in water is treated with an aqueous sodium acetate solution (pH 3.5) for 45 minutes. The title phosphorothioate 20 mer deoxyoligonucleotide having a phosphorothioate monoester attached to the 3′-position of a 3′-terminal adenosine residue is collected after precipitation by addition of ethanol.

### Example 9

### Determination of Initial Cleavage Rates of Duplex Formed Between Antisense Oligodeoxynucleotides and Corresponding Labeled Sense Strand

The initial cleavage rate of heteroduplexes was measured to determine the effect of replacing the 3′-nucleoside of the antisense strand with a phosphorothioate monoester group. The sense strand (SEQ ID NO:3, CGGGTTCGAC CGTAGGCAGT) was 5′-end labeled with ³²P using [γ-³²P]ATP, T4 polynucleotide kinase or alternatively 3′-end labeled with [³²P]pCp using T4 RNA ligase. The labeled sense strand was purified by electrophoresis on a 12% denaturing PAGE, (see; Lima et al., Biochemistry, 1992, 31, 12055). The specific activity of the labeled sense strand was approximately 3000 to 8000 cpm/fmol.

Antisense oligodeoxynucleotide (SEQ ID NO: 1) was prepared to be complementary to and the same number of bases in length as the labeled sense strand. Antisense oligodeoxynucleotide (SEQ ID NO: 2) was prepared identical to SEQ ID NO: 1 with the 3′-deoxynucleoside replaced with a phosphorothioate monoester functional group (SEQ ED NO: 2).

The heteroduplex substrate was prepared in 100 μL containing 20 nM unlabeled oligoribonucleotide (SEQ ID NO: 3), 10⁵ cpm of ³²P labeled oligoribonucleotide (SEQ ID NO: 3), 40 nM complementary oligodeoxynucleotide (either SEQ ID NO: 1 or 2) and hybridization buffer [20 mM tris, pH 7.5, 20 mM KCl]. Reactions were heated at 90° C. for 5 min, cooled to 37° C. and MgCl₂ was added to a final concentration of 1 mM. Hybridization reactions were incubated from 2 to 16 hours at 37° C. and β-mercaptoethanol (BME) was added to a final concentration of 20 mM.

Determinations of Initial Rates (V₀)

The background control was prepared by incubating a 10 μl aliquot of the heteroduplex substrate without human RNase H1 at 37° C. for the duration of the assay. The heteroduplex substrate was digested with 0.5 ng human RNase H1 at 37° C. A 10 μL aliquot of the cleavage reaction was removed at time points ranging from 2 to 120 minutes and quenched by adding 5 μL of stop solution (8 M urea and 120 mM EDTA) and snap-freezing on dry ice. The aliquots were heated at 90° C. for two minutes, resolved in a 12% denaturing polyacrylamide gel and the substrate and product bands were quantitated on a Molecular Dynamics Phosphorimager.

For acid precipitation the 10 μL aliquot of the cleavage reaction was quenched with 90 μL of 0.6 mg/mL yeast tRNA and then precipitated on ice with 100 μL 10% trichloroacetic acid (Sigma, Mo.) for 5 minutes. The sample was centrifuged at 15,000 g, for 5 minutes at 4° C. A 150 μL aliquot of the supernatant was removed and added to 2 mL of scintillation cocktail and the solubilized radioactivity counted in a scintillation counter.

The concentration of converted substrate is calculated by measuring the fraction of substrate converted to product (acid soluble counts or counts for cleavage product bands/total counts) for each time point, multiplying by the substrate concentration and correcting for background ((fraction product×[total substrate])−background). The background values represent the fraction corresponding to the degradation products (counts for non-specific degradation products/total counts). The concentration of the converted product was plotted as a function of time. The initial cleavage rate was obtained from the slope (mole RNA cleaved/min) of the best-fit line for the linear portion of the plot, which comprises, in general <10% of the total reaction. The initial rate line represents data from at least four time points. The time points were selected through iterative testing to obtain a sufficient number of data points within the linear portion of the rate curve.

| SEQ | | | |
|---|---|---|---|
| ID | | | |
| NO: | Vₒ (pM/min) | P | Sequence |
| 1 | 4.48 ± 0.81 | — | 5′-GCCCAAGCTG GCATCCGTCA |
| 2 | 25.91 ± 3.30 | 0.001 | 5′-GCCCAAGCTG GCATCCGTC- |
| | | | PSO₂ |

The results illustrated in the table above show that replacing the 3′terminal nucleoside of SEQ ID NO: 1 with a anionic moiety such as a phosphorothioate monoester moiety increases the rate of cleavage by human RNase H1. As compared to the antisense 20mer the antisense 19mer having the terminal anionic phosphorothioate monoester functional group is cleaved at a rate that is about six times faster (Vₒ=25.91±3.30).

RNase H Initial Rate Determination on the Duplex Formed with 3′-TPT:

Experimental: ³²P Labelling of Oligoribonucleotides: The sense strand was 5′-end labeled with ³²P using [γ-³²P]ATP, T4 polynucleotide kinase, and standard procedures. The labeled RNA was purified by electrophoresis on 12% denaturing PAGE. The specific activity of the labeled oligonucleotide was approximately 6000 cpm/fmol.

Determination of Initial Rates: Hybridization reactions were prepared in 100 μL of reaction buffer [20 mM tris, pH 7.5, 20 mM KCl, 1 mM MgCl₂, 5 mM β-mercaptoethanol] containing 100 nM antisense phosphorothioate oligonucleotide, 50 nM sense oligoribonucleotide, and 100,000 cpm of ³²P labeled sense oligoribonucleotide. Reactions were heated at 90° C. for 5 min. and cooled to 37° C. prior to adding MgCl₂. Hybridization reactions were incubated overnight at 37° C. Hybrids were digested with 0.5 ng human RNase H1 at 37° C. Digestion reactions were analyzed at specific time points in 3 M urea and 20 nM EDTA. Samples were analyzed by trichloroacetic acid assay.

Results and Discussion: The concentration of substrate converted to product was plotted as a function of time. The initial cleavage rate (Vₒ) was obtained from the slope (pM converted substrate per minute) of the best-fit line derived from ≧5 data points within the linear portion (<10% of the total reaction) of the plot. The errors reported were based on three trials and is shown in the table below:

| | | | |
|---|---|---|---|
| Sample | Vₒ (pM/min) | P | Sequence |

| | | | |
|---|---|---|---|
| 2302 | 4.48 ± 0.81 | — | 5′- |
| | | | GCCCAAGCTGGCATCCGTCA |
| 2302-TPT | 25.91 ± 3.30 | 0.001 | 5′- |
| | | | GCCCAAGCTGGCATCCGTC- |
| | | | PSO₂ |

Analysis of the above table shows that the 3′-TPT species behaves better than the parent drug (Vₒ=25.91±3.30) and is approximately six times more potent (P=0.001) than SEQ ID NO:1.

### Example 10

### 5′-thiophosphate-2′-deoxy-2′-fluoro oligonucleotides (SEQ ID NO's: 4-6)

Oligonucleotides with 2′-deoxy-2′-fluoro modifications were synthesized using 2′-deoxy-2′-fluoro-phosphoramidite building blocks (synthesized according to a reported procedure J. Med. Chem, 1993, 36, 831-841). A 0.1 M solution of the respective amidites in anhydrous acetonitrile was used for the synthesis of modified oligonucleotides. For incorporation of a 2′-deoxy-2′-fluro modification, phosphoramidite solutions were delivered in two portions, each followed by a 5 minute coupling wait time. Oxidation of the internucleotide phosphoramidate linkage was carried out using tert-butylhydroperoxide:acetonitrile:water, 10:87:3 with a 10 minute oxidation wait time. For sulfurization a 0.3 M solution of Beaucage reagent in acetonitrile was used. The introduction of a 5′-Phosphate group was achieved with a 0.1 M solution of compound 1a (Glen Research Inc. Virginia, USA) or 2a (J. Med. Chem. 1995, 38, 3941-3950) with a coupling wait time of 10 minutes.

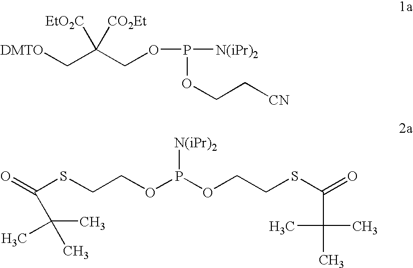

All other steps in the protocol supplied by Millipore were used without any modifications. The observed coupling efficiencies were greater than 97%. The solid support was suspended in saturated methanolic ammonia and kept at room temperature for 24 h to remove the protecting groups from exocyclic amino groups as well as from the phosphate backbone. The crude oligonucleotides were purified by High Performance Liquid Chromatography (HPLC, Waters, C-4, 7.8×300 mm, A=100 mM triethylammonium acetate, pH=6.5-7, B=acetonitrile, 5 to 60% B in 55 Min, Flow 2.5 mL min⁻¹, λ=260 nm). The fractions containing the full-length oligonucleotides were concentrated and adjusted to have a pH of 3.5 with acetic acid and kept at room temperature for 3 hours to remove the dimethoxy trityl group from 5′-end. The oligonucleotides were desalted by HPLC on C-4 column to yield 2′-modified oligonucleotides. Oligonucleotides were characterized by mass spectroscopy and purity was assessed by HPLC and Capillary Gel Electrophoresis. The isolated yields for modified oligonucleotides were 30%.

TABLE 1

5′-thiophosphate-2′-deoxy-2′-Fluoro oligonucle-
otides targeted to siRNA mediated PTEN message

| SEQ ID | |
|---|---|
| NO | Sequence 5′-3′ |
| 4 | 5′ O₂P(S)-O-U*oU*oU*o G*oU*oC*o U*oC*oU*o |
| | G*oG*o U*o C*oC*oU*o |
| | U*oA*oC*o U*oU*3′ |
| 5 | 5′ O₂P(S)-O-A*oA*oA*o C*oA*oG*o A*oG*oA*o |
| | C*oC*o A*o G*oG*oA*o |
| | A*oU*oG*o A*oA*3′ |
| 6 | 5′ O₂P(S)-O-U*sU*sU*s G*sU*sC*s U*sC*sU*s |
| | G*sG*sU*s C*sC*sU*s |
| | U*sA*sC*s U*sU*3′ |

U* = 2′-deoxy-2′-fluorouridine,
A* = 2′-deoxy-2′-fluoroadenosine,
C* = 2′-deoxy-2′-fluorocytidine,
G* = 2′-deoxy-2′-fluoroguanosine,
o = PO,
s = PS

### Example 11

### Synthesis of 5′-thiophosphate RNA (SEQ ID NO's: 7-9) for siRNA Mediated Target Reduction

5′-thiophosphate RNA (SEQ ID NO's: 7-9, Table 2) are synthesized according to the procedure illustrated in example 10 above using commercially available 2′-O-TBDMS ribonucleoside-3′-phosphoramidites and 5′-chemical phosphorylating reagents 1a or 2a.

TABLE 2

5′-thiophosphate RNA targeted to siRNA mediated
PTEN message

| SEQ | |
|---|---|
| ID | |
| NO | Sequence 5′-3′ |
| 7 | 5′ O₂P(S)-O-UoUoUo GoUoCo UoCoUo GoGo Uo |
| | CoCoUo UoAoCo UoU 3′ |
| 8 | 5′ O₂P(S)-O-AoAoAo CoAoGo AoGoAo CoCo Ao |
| | GoGoAo AoUoGo AoA 3′ |
| 9 | 5′ O₂P(S)-O-UsUsUs GsUsCs UsCsUs GsGsUs CsCsUs |
| | UsAsCs UsU 3′ |

o = PO,
s = PS

### Example 12

### Synthesis of 5′-thiophosphate RNA 2′-O-methyl hemimers (SEQ ID NO's: 10-12) for siRNA Mediated Target Reduction

5′-Thiophosphate RNA 2′-O-methyl hemimers (SEQ ID NO's: 10-12, Table 3) are synthesized according to the procedure illustrated in example 10 above using commercially available 2′-O-TBDMS ribonucleoside 3′-phosphoramidites (Glen Research Inc.) and 2′-O-methyl nucleoside phosphoramidites (Glen Research Inc.) and 5′-chemical phosphorylating reagents 1a or 2a.

TABLE 3

5′-thiophosphate RNA 2′-O-methyl hemimers
targeted to siRNA mediated PTEN message

| SEQ | |
|---|---|
| ID | |
| NO | Sequence 5′-3′ |
| 10 | 5′ O₂P(S)-O-UoUoUo GoUoCo UaCoUo GoGo Uo |
| | CoCoU*o U*oA*oC*o U*oU* 3′ |
| 11 | 5′ O₂P(S)-O-AoAoAo CoAoGo AoGoAo CoCo Ao |
| | GoGoAo A*oU*oG*o A*oA* 3′ |
| 12 | 5′ O₂P(S)-O-UsUsUs GsUsCs UsCsUs GsGsUs CsCsUs |
| | U*sA*sC*s U*sU* 3′ |

U* = 2′-O-methyluridine,
A* = 2′-O-methyladenosine,
C* = 2′-O-methylcytidine,
o = PO,
s = PS

### Example 13

### Synthesis of 5′-thiophosphate-RNA-2′-deoxy-2′-fluro hemimers (SEQ ID NO's: 13-15) for siRNA Mediated Target Reduction

5′-Thiophosphate-RNA-2′-deoxy-2′-fluro hemimers (SEQ ID NO's: 13-15, Table 4) are synthesized according to the procedure illustrated in example 10 above using commercially available 2′-O-TBDMS ribonucleoside 3′-phosphoramidites and 2′-deoxy-2′-fluoro nucleoside phosphoramidites (J. Med. Chem. 1993, 36, 831-841) and 5′-chemical phosphorylating reagents 1a or 2a

TABLE 4

5′-Thiophosphate-RNA-2′-deoxy-2′-fluoro hemi-
mers targeted to siRNA mediated PTEN message

| SEQ | |
|---|---|
| ID | |
| NO | Sequence 5′-3′ |
| 13 | 5′ O₂P(S)-O-UoUoUo GoUoCo UoCoUo GoGo Uo |
| | CoCoU*o U*oA*oC*o U*oU* 3′ |
| 14 | 5′ O₂P(S)-O-AoAoAo CoAoGo AoGoAo CoCo Ao GoGoAo |
| | A*oU*oG*o AoA* 3′ |
| 15 | 5′ O₂P(S)-O-UsUsUs GsUsCs UsCsUs GsGsUs CsCsUs |
| | U*sA*sC*s U*sU* 3′ |

U* = 2′-deoxy-2′-fluorouridine,
A* = 2′-deoxy-2′-fluoroadenosine,
C* = 2′-deoxy-2′-fluorocytidine,
G* = 2′-deoxy-2′-fluoroguanosine,
o = PO,
s = PS

### Example 14

### Synthesis of 5′-thiophosphate-2′,5′-RNA (SEQ ID NO's: 16-18) for siRNA Mediated Target Reduction

5′-Thiophosphate 2′,5′-RNA (SEQ ID NO's: 16-18, Table 5) are synthesized according to the procedure illustrated in example 10 above using commercially available 3′-O-TBDMS ribonucleoside 2′-phosphoramdites (Chemgenes, Waltham, Mass. 0254) and 5′-chemical phosphorylating reagents 1a or 2a.

TABLE 5

5′-Thiophosphate 2′,5′-RNA targeted to siRNA
mediated PTEN message

| | |
|---|---|
| SEQ ID NO | Sequence 5′-3′ |

| | |
|---|---|
| 16 | 5′ O₂P(S)-O-U*oU*oU*o G*oU*oC*o U*oC*oU*o |
| | G*oG*o U*o C*oC*oU*o U*oA*oC*o |
| | U*oU* 3′ |
| 17 | 5′ O₂P(S)-O-A*oA*oA*o C*oA*oG*o A*oG*oA*o |
| | C*oC*o A*o G*oG*oA*o |
| | A*oU*oG*o A*oA* 3′ |
| 18 | 5′ O₂P(S)-O-U*sU*sU*s G*sU*sC*s U*sC*sU*s |
| | G*sG*sU*s C*sC*sU*s U*sA*sC*s |
| | U*sU* 3′ |

* = 2′,5′-linkage,
o = PO,
s = PS

### Example 15

### Synthesis of 5′-thiophosphate 2′,5′-DNA for siRNA (SEQ ID NO's:19-21) Mediated Target Reduction

5′-Thiophosphate 2′,5′-DNA (SEQ ID NO's: 19-21, Table 6) are synthesized according to the procedure illustrated in example 10 above using commercially available 3′-deoxy-nucleoside-2′-phosphoramidites (Glen Research Inc, Sterling, Va.) and 5′-chemical phosphorylating reagents 1a or 2a.

TABLE 6

5′-Thiophosphate 2′,5′-DNA targeted to siRNA
mediated PTEN message

| SEQ ID | |
|---|---|
| NO | Sequence 5′-3′ |
| 19 | 5′ d(O₂P(S)-O-U*oU*oU*o G*oU*oC*o U*oC*oU*o |
| | G*oG*o U*o C*oC*oU*o |
| | U*oA*oC*o U*oU*)3′ |
| 20 | 5′ d(O₂P(S)-O-A*oA*oA*o C*oA*oG*o A*oG*oA*o |
| | C*oC*o A*o G*oG*oA*o |
| | A*oU*oG*o A*oA*)3′ |
| 21 | 5′ d(O₂P(S)-O-U*sU*sU*s G*sU*sC*s U*sC*sU*s |
| | G*sG*sU*s C*sC*sU*s U*sA*sC*s |
| | U*sU*) 3′ |

* = 2′,5′-linkage,
o = PO,
s = PS

### Example 16

### 5′-Deoxy-5′-thiophosphoricacid-O,O′-bis-(2-cyanoethyl)ester-2′-O-tert-butyldimethyl silyl-3′-(2-cyanoethyl)-N,N′-diisopropylphosphoramidite 25a

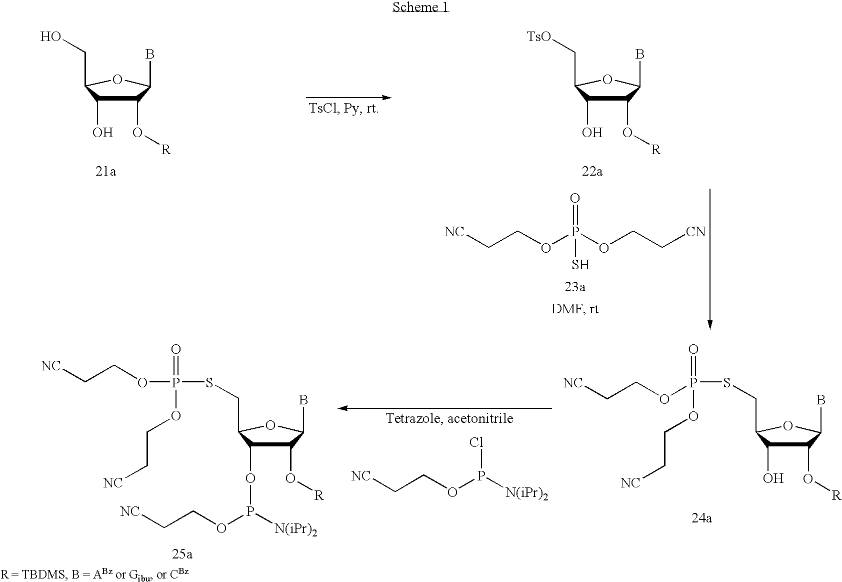

Compound 21a is synthesized as reported (Can. J. Chem. 1982, 60, 1106-1113). Tosylation of compound 21a at 5′ position in pyridine and p-tolunesulfonyl chloride give 22a. Compound 22a is treated with 23a (Proc. Natl. Acad. Sci. U S. A, 1988, 85, 1349-1353) in DMF at room temperature to yield 24a. Compound 24a is converted into 3′-phosphoramidite 25a by treating with 2-cyanoethyl diisopropylchlorophosphoramidite and tetrazole in acetonitrile at room temperature.

### Example 17

### Synthesis of 5′-deoxy-5′-thiophosphoricacid-RNA (SEQ ID NO's: 22-24) for siRNA Mediated Target Reduction

5′-Deoxy-5′-thiophosphoricacid-RNA (SEQ ID NO's: 22-24, Table 7) are synthesized according to the procedure illustrated in example 10 above using commercially available 2′-O-TBDMS ribonucleoside 3′-phosphoramidites and phosphoramidite 25a

TABLE 7

5′-Deoxy-5′-thiophosphoricacid-RNA targeted to
siRNA mediated PTEN message

| SEQ | |
|---|---|
| ID | |
| NO | Sequence 5′-3′ |
| 22 | 5′ O₂P(O)-S-UoUoUo GoUoCo UoCoUo GoGo Uo CoCoUo |
| | UoAoCo UoU 3′ |
| 23 | 5′ O₂P(O)-S-AoAoAo CoAoGo AoGoAo CoCo Ao GoGoAo |
| | AoUoGo AoA 3′ |
| 24 | 5′ O₂P(O)-S-UsUsUs GsUsCs UsCsUs GsGsUs CsCsUs |
| | UsAsCs UsU 3′ |

o = PO,
s = PS

### Example 18

### Synthesis of 5′-deoxy-5′-thiophosphoricacid-RNA-2′-O-methyl hemimers (SEQ ID NO's: 25-27) for siRNA Mediated Target Reduction

5′-Thiophosphate RNA 2′-O-methyl hemimers (SEQ ID NO's: 25-27, Table 8) are synthesized according to the procedure illustrated in example 10 above using commercially available 2′-O-TBDMS ribonucleoside 3′-phosphoramidites (Glen Research Inc.) and 2′-O-methyl nucleoside phosphoramidites (Glen Research Inc.) and phosphoramidite 25a

TABLE 8

5′-dDoxy-5′-Thiophosphoricacid RNA 2′-O-methyl
hemimers targeted to siRNA mediated PTEN
message

| SEQ | |
|---|---|
| ID | |
| NO | Sequence 5′-3′ |
| 25 | 5′ O₂P(O)-S-UoUoUo GoUoCo UoCoUo GoGo Uo |
| | CoCoU*o U*oA*oC*oU*oU* 3′ |
| 26 | 5′ O₂P(O)-S-AoAoAo CoAoGo AoGoAo CoCo Ao GoGoAo |
| | A*oU*oG*o A*oA* 3′ |
| 27 | 5′ O₂P(O)-S-UsUsUs GsUsCs UsCsUs GsGsUs CsCsUs |
| | U*sA*sC*s U*sU* 3′ |
| U* = 2′-O-methyluridine, | |
| A* = 2′-O-methyladenosine, | |
| C* = 2′ O-methylcytidine, | |
| o = PO, | |

s = PS

### Example 19

### Synthesis of 5′-deoxy-5′-thiophosphoricacid-RNA-2′-deoxy-2′-fluoro hemimers (SEQ ID NO's: 28-30) for siRNA MEDIATED TARGET REDUCTION

5′-Thiophosphate RNA 2′-deoxy-2′-fluro hemimers (SEQ ID NO's: 28-30, Table 9) are synthesized according to the procedure illustrated in example 10 above using commercially available 2′-O-TBDMS ribonucleoside 3′-phosphoramidites and 2′-deoxy-2′-fluoro nucleoside phosphoramidites (J. Med. Chem. 1993, 36,831-841) and phosphoramidite 25a.

TABLE 9

5′-Deoxy-5′-thiophosphoricacid-RNA-2′-deoxy-2′-
fluoro hemimers targeted to siRNA mediated PTEN
message

| SEQ | |
|---|---|
| ID | |
| NO | Sequence 5′-3′ |
| 28 | 5′ O₂P(O)-S-UoUoUo GoUoCo UoCoUo GoGo Uo |
| | CoCoU*o U*oA*oC*o U*oU* 3′ |
| 29 | 5′ O₂P(O)-S-AoAoAo CoAoGo AoGoAo CoCo Ao GoGoAo |
| | A*oU*oG*o A*oA* 3′ |
| 30 | 5′ O₂P(O)-S-UsUsUs GsUsCs UsCsUs GsGsUs CsCsUs |
| | U*sA*sC*s U*sU* 3′ |

U* = 2′-deoxy-2′-fluorouridine,
A* = 2′-deoxy-2′-fluoroadenosine,
C* = 2′-deoxy-2′-fluorocytidine,
G* = 2′-deoxy-2′-fluoroguanosine,
o = PO,
s = PS

### Example 20

### Synthesis of 5′-deoxy-5′-thiophosphoricacid 2′,5′-RNA (SEQ ID NO's: 31-33) for siRNA Mediated Target Reduction

5′-Deoxy-5′-thiophosphoricacid 2′,5′-RNA (SEQ ID NO's: 31-33, Table 10) are synthesized according to the procedure illustrated in example 10 above using commercially available 2′-O-TBDMS ribonucleoside 3′-phosphoramidites (Chemgenes, Waltham, Mass. 0254) and phosphoramidite 25a.

TABLE 10

5′-Thiophosphate RNA 2′-O-methyl hemimers
targeted to siRNA mediated PTEN message

| | |
|---|---|
| SEQ ID NO | Sequence 5′-3′ |

| | |
|---|---|
| 31 | 5′ O₂P(O)-S-U*oU*oU*o G*oU*oC*o U*oC*oU*o |
| | G*oG*o U*o C*oC*oU*o U*oA*oC*o |
| | U*oU* 3′ |
| 32 | 5′ O₂P(O)-S-A*oA*oA*o C*oA*oG*o A*oG*oA*o |
| | C*oC*o A*o G*oG*oA*o |
| | A*oU*oG*o A*oA* 3′ |
| 33 | 5′ O₂P(O)-S-U*sU*sU*s G*sU*sC*s U*sC*sU*s |
| | G*sG*sU*s C*sC*sU*s U*sA*sC*s |
| | U*sU* 3′ |

* = 2′,5′-linkage,
o = PO,
s = PS

### Example 21

### Synthesis of 5′-deoxy-5′-thiophosphoricacid 2′,5′-DNA (SEQ ID NO's: 34-36) for siRNA Mediated Target Reduction

5′-Deoxy-5′-thiophosphoricacid 2′,5′-DNA (SEQ ID NO's: 34-36, Table 11) are synthesized according to the procedure illustrated in example 10 above using commercially available 3′-deoxy-nucleoside-2′-phosphoramidites (Glen Research Inc, Sterling, Va.) and phosphoramidite 25a.

TABLE 11

5′-thiophosphate 2′,5′-DNA targeted to siRNA
mediated PTEN message

| SEQ ID | |
|---|---|
| NO | Sequence 5′-3′ |
| 34 | 5′d(O₂P(O)-S-U*oU*oU*o G*oU*oC*o U*oC*oU*o |
| | G*oG*o U*o C*oC*oU*o |
| | U*oA*oC*o U*oU*)3′ |
| 35 | 5′ d(O₂P(O)-S-A*oA*oA*o C*oA*oG*o A*oG*oA*o |
| | C*oC*o A*o G*oG*oA*o |
| | A*oU*oG*o A*oA*) 3′ |
| 36 | 5′ d(O₂P(O)-S-U*sU*sU*s G*sU*sC*s U*sC*sU*s |
| | G*sG*sU*s C*sC*sU*s |
| | U*sA*sC*s U*sU*) 3′ |

* = 2′,5′-linkage,
o = PO,
s = PS

### Example 22

### 5′-Deoxy-5′-dithiophosphoricacid-O,O′-bis-(2-cyano-ethyl)ester-2′-O-tert-butyldimethylsilyl-3′-(2-cyanoethyl)-N,N′-diisopropylphosphoramidite 43a

Compound 41a is synthesized as reported (JP 92-63802, 1993). Compound 22a is treated with 41a in DMF at room temperature to yield 42a. Phoshitylation of compound 42a at 3′-position with 2-(cyanoethyl)-N,N-diisopropylphosphoramidite in acetonitrile in presence of tetrazole give compound 43a.

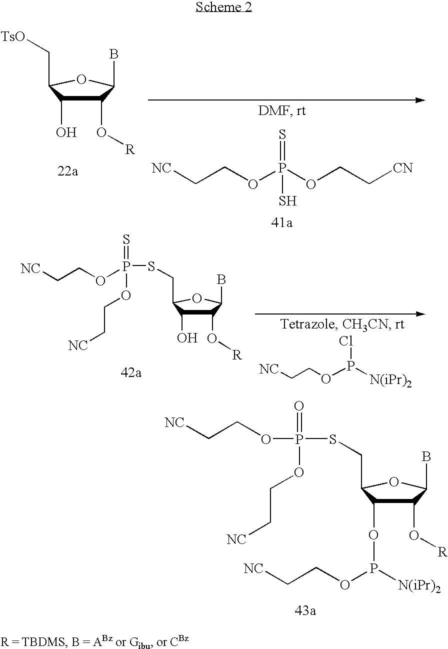

### Example 23

### Synthesis of 5′-deoxy-5′-dithiophosphoricacid-RNA (SEQ ID NO's: 37-39) for siRNA Mediated Target Reduction

5′-Dithiophosphate-RNA (SEQ ID NO's: 37-39, Table 12) are synthesized according to the procedure illustrated in example 10 above using commercially available 2′-O-TBDMS ribonucleoside 3′-phosphoramidites and phosphoramidite 43a.

TABLE 12

5′-deoxy-5′-dithiophosphoricacid-RNA targeted
to siRNA mediated PTEN message

| SEQ | |
|---|---|
| ID | |
| NO | Sequence 5′-3′ |
| 37 | 5′ O₂P(S)-S-UoUoUo GoUoCo UoCoUo GoGo Uo CoCoUo |
| | UoAoCo UoU 3′ |
| 38 | 5′ O₂P(S)-S-AoAoAo CoAoGo AoGoAo CoCo Ao GoGoAo |
| | AoUoGo AoA 3′ |
| 39 | 5′ O₂P(S)-S-UsUsUs GsUsCs UsGsUs GsGsUs CsCsUs |
| | UsAsCs UsU 3′ |

o = PO,
s = PS

### Example 24

### Synthesis of 5′-deoxy-5′-dithiophosphoricacid-RNA-2′-O-methyl hemimers (SEQ ID NO's: 40-42) for siRNA Mediated Target Reduction

5′-Deoxy-5′-dithiophosphoricacid-RNA 2′-O-methyl hemimers (SEQ ID NO's: 40-42, Table 13) are synthesized according to the procedure illustrated in example 10 above using commercially available 2′-O-TBDMS ribonucleoside 3′-phosphoramidites (Glen Research Inc.) and 2′-O-methyl nucleoside phosphoramidites (Glen Research Inc.) and phosphoramidite 43a.

TABLE 13

5′-deoxy-5′-dithiophosphoricacid RNA 2′-O-
methyl hemimers targeted to siRNA mediated PTEN
message

| SEQ | |
|---|---|
| ID | |
| NO | Sequence 5′-3′ |
| 40 | 5′ O₂P(S)-S-UoUoUo GoUoCo UoCoUo GoGo Uo |
| | CoCoU*o U*oA*oC*o U*oU* 3′ |
| 41 | 5′ O₂P(S)-S-AoAoAo CoAoGo AoGoAo CoCo Ao GoGoAo |
| | A*oU*oG*o A*oA* 3′ |
| 42 | 5′ O₂P(S)-S-UsUsUs GsUsCs UsCsUs GsGsUs CsCsUs |
| | U*sA*sC*s U*sU* 3′ |

U* = 2′-O-methyluridine,
A* = 2′-O-methyladenosine,
C* = 2′-O-methylcytidine,
o = PO,
s = PS

### Example 25

### Synthesis of 5′-deoxy-5′-dithiophosphoricacid-RNA-2′-deoxy-2′-fluro hemimers (SEQ ID NO's: 43-45) for siRNA Mediated Target Reduction

5′-Deoxy-5′-dithiophosphoricacid-RNA-2′-deoxy-2′-fluoro hemimers (SEQ ID NO's: 43-45, Table 14) are synthesized according to the procedure illustrated in example 10 above using commercially available 2′-O-TBDMS ribonucleoside 3′-phosphoramidites and 2′-deoxy-2′-fluoro nucleoside phosphoramidites (J. Med. Chem. 1993, 36, 831-841) and phosphoramidite 43a.

TABLE 14

5′-Deoxy-5′-dithiophosphoricacid-RNA 2′-deoxy-
2′-fluoro hemimers targeted to siRNA mediated
PTEN message

| SEQ | |
|---|---|
| ID | |
| NO | Sequence 5′-3′ |
| 43 | 5′ O₂P(S)-S-UoUoUo GoUoCo UoCoUo GoGo Uo |
| | CoCoU*o U*oA*oC*o U*oU* 3′ |
| 44 | 5′ O₂P(S)-S-AoAoAo CoAoGo AoGoAo CoCo Ao GoGoAo |
| | A*oU*oG*oA*oA* 3′ |
| 45 | 5′ O₂P(S)-S-UsUsUs GsUsCs UsCsUs GsGsUs CsCsUs |
| | U*sA*sC*s U*sU* 3′ |

U* = 2′-deoxy-2′-fluorouridine,
A* = 2′-deoxy-2′-fluoroadenosine,
C* = 2′-deoxy-2′-fluorocytidine,
G* = 2′-deoxy-2′-fluoroguanosine,
o = PO,
s = PS

### Example 26

### Synthesis of 5′-deoxy-5′-dithiophosphoricacid-2′,5′-RNA (SEQ ID NO's: 46-48) for siRNA Mediated Target Reduction

5′-Deoxy-5′-dithiophosphoricacid-2′,5′-RNA (SEQ ID NO's: 46-48, Table 13) are synthesized according to the procedure illustrated in example 10 above using commercially available 2′-O-TBDMS ribonucleoside 3′-phosphoramidites (Chemgenes, Waltham, Mass. 0254) and phosphoramidite 31a.

TABLE 15

5′ -deoxy-5′-dithiophosphoricacid-RNA-2′-O-
methyl hemimers targeted to siRNA mediated PTEN
message

| | |
|---|---|
| SEQ ID NO | Sequence 5′-3′ |

| | |
|---|---|
| 46 | 5′ O₂P(S)-S-U*oU*oU*o G*oU*oC*o U*oC*oU*o |
| | G*oG*o U*o C*oC*oU*o |
| | U*oA*oC*oU*oU* 3′ |
| 47 | 5′ O₂P(S)-S-A*oA*oA*o C*oA*oG*o A*oG*oA*o |
| | C*oC*o A*o G*oG*oA*o |
| | A*oU*oG*o A*oA* 3′ |
| 48 | 5′ O₂P(S)-S-U*sU*sU*s G*sU*sC*s U*sC*sU*s |
| | G*sG*sU*s C*sC*sU*s U*sA*sC*s |
| | U*sU* 3′ |

* = 2′,5′-linkage,
o = PO,
s = PS

### Example 27

### Synthesis of 5′-deoxy-5′-dithiophosphoricacid-2′,5′-DNA (SEQ ID NO's: 49-51) for siRNA Mediated Target Reduction

5′-Deoxy-5′-dithiophosphoricacid 2′,5′-DNA (SEQ ID NO's: 49-51, Table 16) are synthesized according to the procedure illustrated in example 10 above using commercially available 3′-deoxy-nucleoside-2′-phosphoramidites (Glen Research Inc, Sterling, Va.) and phosphoramidite 31a.

TABLE 16

5′-deoxy-5′-dithiophosphoricacid-2′,5′-DNA tar-
geted to siRNA PTEN message

| SEQ ID | |
|---|---|
| NO | Sequence 5′-3′ |
| 49 | 5′ d(O₂P(S)-S-U*oU*oU*o G*oU*oC*o U*oC*oU*o |
| | G*oG*o U*o C*oC*oU*o |
| | U*oA*oC*o U*oU*)3′ |
| 50 | 5′ d(O₂P(S)-S-A*oA*oA*o C*oA*oG*o A*oG*oA*o |
| | C*oC*o A*o G*oG*oA*o |
| | A*oU*oG*o A*oA*) 3′ |
| 51 | 5′ d(O₂P(S)-S-U*sU*sU*s G*sU*sC*s U*sC*sU*s |
| | G*sG*sU*s C*sC*sU*s U*sA*sC*s |
| | U*sU*) 3′ |

* = 2′,5′-linkage,
o = PO,
s = PS

### Example 30

### Oligonucleotide and Oligonucleoside Synthesis

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄OAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published PCT applications PCT/U.S. 94/00902 and PCT/U.S. 93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 31

### RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dall, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense compounds (RNA oligonucleotides) of the present invention can be synthesized by the methods herein or purchased from Dharmacon Research, Inc (Lafayette, Colo.). Once synthesized, complementary RNA antisense compounds can then be annealed by methods known in the art to form double stranded (duplexed) antisense compounds. For example, duplexes can be formed by combining 30 μl of each of the complementary strands of RNA oligonucleotides (50 uM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense compounds can be used in kits, assays, screens, or other methods to investigate the role of a target nucleic acid.

### Example 32

### Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

### [2′-O-Me]-[2′-deoxy]-[2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

### [2′-O-(2-Methoxyethyl)]-[2′-deoxy]-[2′-O-(Methoxyethyl)] Chimeric Phosphorothioate Oligonucleotides

[2′-O-(2-methoxyethyl)]-[2′-deoxy]-[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

### [2′-O-(2-Methoxyethyl)Phosphodiester—]—[2′-deoxy Phosphorothioate]—2′-O-(2-Methoxyethyl) Phosphodiester] Chimeric Oligonucleotides

[2′-O-(2-methoxyethyl phosphodiester]—[2′-deoxy phosphorothioate]—[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 33

### Design and Screening of Duplexed Antisense Compounds Targeting a Target

In accordance with the present invention, a series of nucleic acid duplexes comprising the antisense compounds of the present invention and their complements can be designed to target a target. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG and having a two-nucleobase overhang of deoxythymidine (dT) would have the following structure:

| | |
|---|---|
| cgagaggcggacgggaccgTT | Antisense |
| \|\|\|\|\|\|\|\|\|\|\|\|\|\|\|\|\|\|\| | Strand |
| TTgctctccgcctgccctggc | Complement |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is. 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense compounds are evaluated for their ability to modulate a target expression.

When cells reached 80% confluency, they are treated with duplexed antisense compounds of the invention. For cells grown in 96-well plates, wells are washed once with 200 μL OPTI-MBM-1 reduced-serum medium (Gibco BRL) and then treated with 130 μL of OPTI-MEM-1 containing 12 μg/mL LIPOFECTIN (Gibco BRL) and the desired duplex antisense compound at a final concentration of 200 nM. After 5 hours of treatment, the medium is replaced with fresh medium. Cells are harvested 16 hours after treatment, at which time RNA is isolated and target reduction measured by RT-PCR.

### Example 34

### Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32+/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 35

### Oligonucleotide Synthesis—96 Well Plate Format

Oligonucleotides were synthesized via solid phase P(M) phosphoramidite chemistry on all automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 36

### Oligonucleotide Analysis—96-Well Plate Format

The concentration of oligonucleotide in each well was assessed by dilution of samples and UV absorption spectroscopy. The fill-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 37

### Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF cells:

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

Treatment with Antisense Compounds:

When cells reached 65-75% confluency, they were treated with oligonucleotide. For cells grown in 96-well plates, wells were washed once with 100 μL OPTI-MEM™-1 reduced-serum medium Invitrogen Corporation, Carlsbad, Calif.) and then treated with 130 μL of OPTI-MEM™-1 containing 3.75 μg/mL LIPOFECTIN™ Invitrogen Corporation, Carlsbad, Calif.) and the desired concentration of oligonucleotide. Cells are treated and data are obtained in triplicate. After 4-7 hours of treatment at 37° C., the medium was replaced with fresh medium. Cells were harvested 16-24 hours after oligonucleotide treatment

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO: 52) which is targeted to human H-ras, or ISIS 18078, (GTGCGCGCGAGCCCGAAATC, SEQ ID NO: 53) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO: 54, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### Example 38

### Analysis of Oligonucleotide Inhibition of a Target Expression

Antisense modulation of a target expression can be assayed in a variety of ways known in the art. For example, a target mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. The preferred method of RNA analysis of the present invention is the use of total cellular RNA as described in other examples herein. Methods of RNA isolation are well known in the art. Northern blot analysis is also routine in the art. Real-time quantitative (ACR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of a target can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### Example 39

### Design of Phenotypic Assays and In Vivo Studies for the Use of a Target Inhibitors

Phenotypic Assays

Once a target inhibitors have been identified by the methods disclosed herein, the compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of a target in health and disease. Representative phenotypic assays, which can be purchased from any one of several commercial vendors, include those for determining cell viability, cytotoxicity, proliferation or cell survival Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with a target inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the geneotype of the cell (measurement of the expression of one or more of the genes of the cell) after treatment is also used as an indicator of the efficacy or potency of the a target inhibitors. Hallmark genes, or those genes suspected to be associated with a specific disease state, condition, or phenotype, are measured in both treated and untreated cells.

In Vivo Studies

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study.

To account for the psychological effects of receiving treatments, volunteers are randomly given placebo or a target inhibitor. Furthermore, to prevent the doctors from being biased in treatments, they are not informed as to whether the medication they are administering is a a target inhibitor or a placebo. Using this randomization approach, each volunteer has the same chance of being given either the new treatment or the placebo.

Volunteers receive either the a target inhibitor or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding a target or a target protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and a target inhibitor treatment. In general, the volunteers treated with placebo have little or no response to treatment, whereas the volunteers treated with the a target inhibitor show positive trends in their disease state or condition index at the conclusion of the study.

### Example 40

### RNA Isolation

Poly(A)+ mRNA Isolation

Poly(A)+ mRNA was isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 41

### Real-Time Quantitative PCR Analysis of a Target mRNA Levels

Quantitation of a target mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PEApplied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5× ROX dye) to 96-well plates containing 30 mL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374). In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

Probes and are designed to hybridize to a human a target sequence, using published sequence information.

### Example 42

### Northern Blot Analysis of a Target mRNA Levels

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESSCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human a target, a human a target specific primer probe set is prepared by PCR To normalize for variations in loading and transfer efficiency membranes are stripped and probed for human glyceraldehyde-3-phosphate dehydrogenase (GAPDH) RNA (Clontech, Palo Alto, Calif.).

Hybridized membranes were visualized and quantitated using a PHOSPHORBIMAGER™ and IMAGEQUANT™ Software V3.3 (Molecular Dynamics, Sunnyvale, Calif.). Data was normalized to GAPDH levels in untreated controls.

### Example 43

Antisense inhibition of human a target expression by oligonucleotides In Accordance with the present invention, a series of compounds are designed to target different regions of the human target RNA. The compounds are analyzed for their effect on human target mRNA levels by quantitative real-time PCR as described in other examples herein. Data are averages from three experiments. The target regions to which these preferred sequences are complementary are herein referred to as “preferred target segments” and are therefore preferred for targeting by compounds of the present invention. The sequences represent the reverse complement of the preferred antisense compounds.

As these “preferred target segments” have been found by experimentation to be open to, and accessible for, hybridization with the antisense compounds of the present invention, one of skill in the art will recognize or be able to ascertain, using no more than routine experimentation, further embodiments of the invention that encompass other compounds that specifically hybridize to these preferred target segments and consequently inhibit the expression of a target.

According to the present invention, antisense compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 44

### Western Blot Analysis of a Target Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to a target is used, with a radiolabeled or fluorescently labeled secondary antibody directed against the primary antibody species. Bands are visualized using a PHOSPHORIMAGER™ (Molecular Dynamics, Sunnyvale Calif.).

### Example 45

### Preparation of 3′-modified oligonucleotides (phosphate, phosphorothioate, methyl phosphonate, and methyl phosphorothionate)

A 19-mer phosphorothioate oligodeoxyribonucleotide targeted to BCLx expression inhibition was selected for modification. The 3′-terminus of this sequence was prepared having modifications a-h illustrated below as well as the control sequence having modification i at the 3′-terminus.

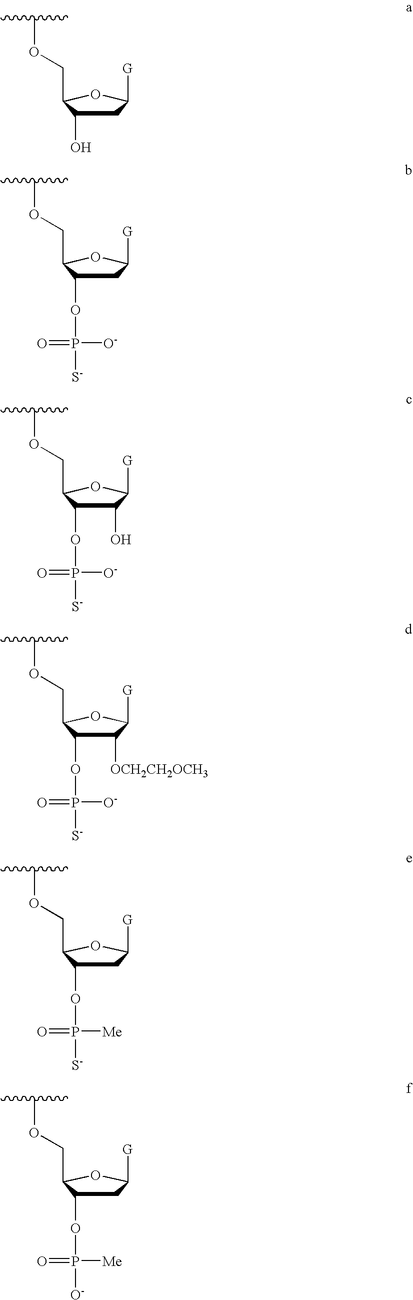

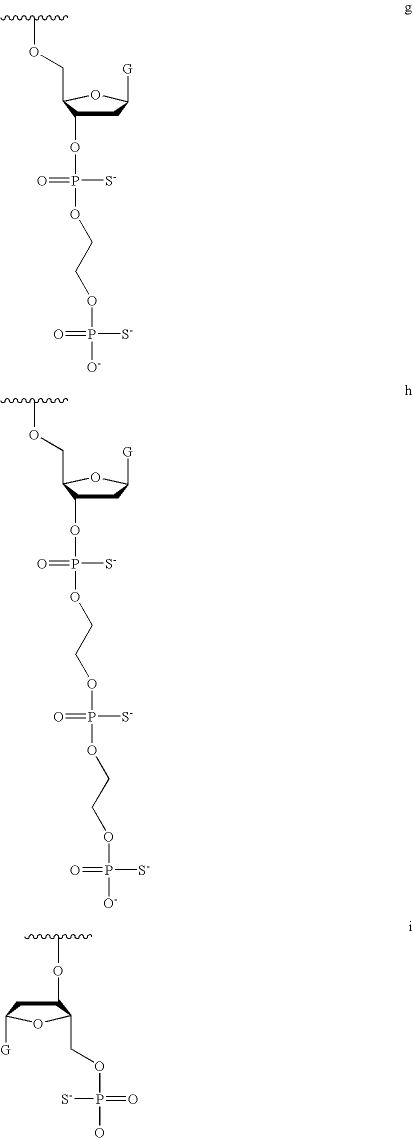

FIG. 1. 3′-modifications synthesized for evaluation

TABLE 1

Oligonucleotides used for the investigation.

| SEQ | | 3′-terminal |
|---|---|---|
| ID | | modification |
| NO | sequence | (Gₓ) |
| 55 | PS[d(CTA-CGC-TTT-CCA-CGC-ACA-G)] | a |
| 56 | PS[d(CTA-CGC-TTT-CCA-CGC-ACA-Gₓ)] | b |
| 57 | | c |
| 58 | | d |
| 59 | | e |
| 60 | | f |
| 61 | | g |
| 62 | | h |
| 63 | | i |

Synthesis and Characterization of Oligonucleotides Containing Modifications:

Although commercially available glass supports (CPG) containing Phosphate-ON reagent are available, the corresponding version of Primer Support PS200 is not commercially available. The modified oligonucleotides were prepared starting with standard thymidine-loaded PS200 solid support to which was coupled a Phosphate-ON phosphoramidite. Then the oligonucleotide synthesis was performed on this modified support. Afterward, incubation with concentrated aqueous ammonium hydroxide liberated the phosphorothioate oligonucleotide from the support with formation of the thymidine-5′-phosphate monoester. This nucleoside monomer is easily removed during reverse phase HPLC purification. Alternatively, another method of synthesis of the 3′-phosphate/phosphorothioate monoester utilizes a “trimethyl lock” based molecule. This derivatized solid support has been used in synthesis of several 3′-negatively charged oligonucleotides (see Cheruvallath, Z. S.; Cole, D. L.; Ravikumar, V. T. Bioorg. Med. Chem. Lett., 2003, 13, 281.)

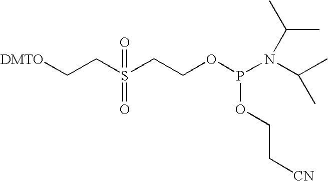

Phosphate-ON Reagent

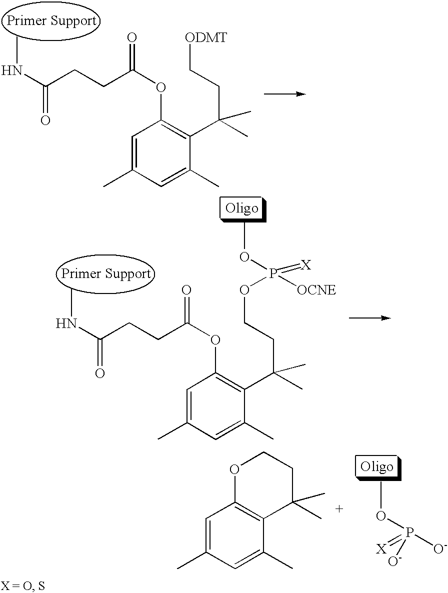

Novel solid support used for synthesis of 3′-Phosphorothioate derivatives

Crude DMT-on oligomer was purified by reverse phase IPLC under standard conditions, fractionated and the desired fractions were pooled. Detritylation was performed following standard protocols, and the oligomer was precipitated and lyophilized to afford a colorless amorphous powder. The purified oligonucleotides were analyzed by capillary gel eletrophoresis (CGE, Table 17), ³¹P NMR and eletrospray quadrupole mass spectroscopy were consistent with the expected sequences.

TABLE 17

| | |
|---|---|
| HPLC | |
| retention | mass |

| SEQ ID NO | time, min.^{a} | calculated | found |
|---|---|---|---|
| 55 | 21.02 | 5997.20 | 5997.26 |
| 56 | 20.94 | 6093.82 | 6093.37 |
| 57 | 20.94 | 6108.97 | 6109.34 |
| 58 | 20.96 | 6167.72 | 6167.41 |
| 59 | 21.05 | 6090.89 | 6091.43 |
| 60 | 20.88 | 6075.13 | 6075.30 |
| 61 | 20.97 | 6231.95 | 6232.36 |
| 62 | 20.94 | 6371.88 | 6371.36 |
| 63 | 20.91 | 6093.22 | 6093.71 |

Table 2. Characteriscis of DNA analogues possessing 3′-terminal charge
^{a}Phenomenex, C18, 4.6 × 250 mm, A = 100 mM triethylammonium acetate, pH 7, flow rate 1.0 mL min⁻¹, λ = 260 nm, B = acetonitrile, 0-40% B from 0 to 25 min, 40% B from 25 to min, 100% B from 30 to 39 min, 100% A from 39 min to 45 min.

³²P Labelling of Oligoribonucleotides: The sense strand was 5′-end labeled with ³²P using [γ-³²P]ATP, T4 polynucleotide kinase, and standard procedures (see Puglisi, J. D.; Tinoco, I. Jr. Methods Enzymol., 1989, 180, 304.) The labeled RNA was purified by electrophoresis on 12% denaturing PAGE. The specific activity of the labeled oligonucleotide was approximately 6000 cpm/fmol.

Determination of Initial Rates: Hybridization reactions were prepared in 100 μL of reaction buffer [20 mM tris, pH 7.5, 20 mM KCl, 1 mM MgCl₂, 5 mM β-mercaptoethanol] containing 100 nM antisense phosphorothioate oligonucleotide, 50 nM sense oligoribonucleotide, and 100,000 CPM of 32 labeled sense oligoribonucleotide. Reactions were heated at 90° C. for 5 min. and cooled to 37° C. prior to adding MgCl₂. Hybridization reactions were incubated overnight at 37° C. Hybrids were digested with 0.5 ng human RNase H1 at 37° C. (see Petersheim, M.; Turner, D. H. Biochemistry, 1983, 22, 256.) Digestion reactions were analyzed at specific time points in 3 M urea and 20 nM EDTA. Samples were analyzed by trichloroacetic acid assay (Ausubel, F. M.; Brent, R.; Kingston, R. E.; Moore, D. D.; Seidman, J. G.; Smith, J. A.; Struhl, K. in Current Protocols in Molecular Bilogy, 1989, John Wiley, New York.) The concentration of substrate converted to product was plotted as a function of time. The initial cleavage rate (Vₒ) was obtained from the slope (pM converted substrate per minute) of the best-fit line derived from ≧5 data points within the linear portion (<10% of the total reaction) of the plot (see Wu, H. J.; Lima, W. F.; Crooke, S. T. Antisense & Nucleic Acid Drug Dev., 1998, 8, 53.) The errors reported were based on three trials and is shown below the table:

| SEQ ID NO | V₀ (pM/min) | P |
|---|---|---|
| 55 | 869 ± 0.953 | — |
| 56 | 850 ± 0.965 | 0.728 |
| 57 | 564 ± 0.937 | 0.009 |
| 58 | 569 ± 0.936 | 0.008 |
| 59 | 1016 ± 0.966 | 0.201 |
| 60 | 982 ± 0.944 | 0.264 |
| 61 | 813 ± 0.963 | 0.049 |
| 62 | 793 ± 0.955 | 0.002 |
| 63 | 792 ± 0.935 | 0.012 |

Rate of cleavage of duplex formed with oligonucleotides containing modifications was observed to be comparable to the rate for the 3′-phosphorothioate monoester modified oligonucleotide.

Experimental: Anhydrous acetonitrile (water content <0.001%) was purchased from Burdick and Jackson (Muskegon, MED. 5′-O-Dimethoxytrityl-3′-N,N-diisopropylaminoe-3′-O-(2-cyanoethyl) phosphoramidites (T, dAbz, dCbz, dGibu) were purchased from Amersham Pharmacia Biotech, Milwaukee, Wis. Methyl phosphoramidite, ethylene glycol amidite, inverted amidite and Phosphate-ON reagent were purchased from ChemGenes, MA. Toluene was purchased from Gallade, Escondido, Calif. All other reagents and dry solvents were purchased from Aldrich and used without further purification. Primer support PS200 was purchased from Amersham Pharmacia Biotech, Uppsala, Sweden. 1H-Tetrazole was purchased from American International Chemical, Natick, Mass. Phenylacetyl disulfide (PADS) was purchased from H. C. Brown Laboratories, Mumbai, India.

31P NMR spectra were recorded on a Unity-200 spectrometer (Varian, Palo Alto, Calif.) operating at 80.950 MHz. Capillary gel electrophoresis was performed on a eCAP ssDNA 100 Gel Capillary (47 cm) on a P/ACE System 5000 using Tris/borate/7 M urea buffer (all Beckman), running voltage 14.1 kV, temperature 40° C. For synthesis of 2, the support-bound DMT-on oligonucleotide was first treated with triethylamine:acetonitrile (1:1, v/v) at room temperature for 8 h, then treated with Et3N-3HF for 7 h at room temperature and then incubated with ammonium hydroxide in the usual manner. Typical procedure for solid supported synthesis of compounds:

All syntheses were performed on a Pharmacia OligoPilot II DNA/RNA synthesizer using β-cyanoethyl phosphoramidite synthons (2.5 equivalents, 0.2M in CH₃CN). 1H-Tetrazole (0.45M in CH₃CN) was used as activator and phenylacetyl disulfide (PADS) (0.2M in 3-picoline-CH₃CN 1:1, v/v) as sulfur transfer reagent. Capping reagents were made to the recommended Pharmacia receipe: Cap A: N-methylimidazole-CH₃CN(1:4v/v), Cap B: acetic anhydride-pyridine-CH₃CN (2:3:5, v/v/v). Pharmacia HL30 T Primer support (loading 94 μmole/gram) was used in all experiments. Amidite and tetrazole solutions were prepared using anhydrous CH₃CN (ca 10 ppm) and were dried further by addition of activated 4 Å molecular sieves (˜50 g/L). 5′-Phosphate-ON reagent was used as a 0.2M solution (2.0 equivalents) in CH₃CN. To introduce the 3′-terminal charge, the commercially available 5′-phosphate-ON reagent was first coupled to the T Primer solid support, then the oligonucleotide constructed. At the end of each synthesis, DMT-on oligonucleotide bound to support was transferred to a 500 mL pyrex glass bottle and treated with CH₃CN:Et₃N (1:1, v/v, 400 mL) at room temperature overnight. The support was filtered and taken up in a 250 mL Pyrex glass bottle. Concentrated aqueous ammonium hydroxide (400 mL) was added and incubated in an oven at 55° C. for 18 h. The bottle was then cooled to room temperature and the solid filtered on a sintered glass funnel. The support was washed with water (250 mL), the combined solution concentrated by rotary evaporator. Triethylamine (4 mL) was added and the product was stored in a refrigerator. Details of the synthesis cycle are given in the Table below:

| | | Volume | Time |
|---|---|---|---|
| tep | Reagent | (ml) | (min) |
| Detritylation | 10% dichloroacetic acid/toluene | 72 | 1.5 |
| Coupling | Phosphoramidite (0.2M), 1H-tetrazole | 10, 15 | 5 |
| | (0.45 m) in acetonitrile | | |
| Sulfurization | Phenylacetyl disulfide (0.2M) in | 36 | 3 |
| | 3-picoline-CH₃CN (1:1, v/v) | | |
| Capping | Ac₂O/pyridine/CH₃CN, NMI/CH₃CN | 24, 24 | 2 |

Synthesis parameters of cycle used on Pharmacia OligoPilot II synthesizer

HPLC Analysis and Purification of Oligonucleotides:

Analysis and purification of oligonucleotides by reversed phase high performance liquid chromatography (RP-HPLC) was performed on a Waters Novapak C₁₈ column (3.9x300 mm) using a Waters HPLC system (600E System Controller, 996 Photodiode Array Detector, 717 Autosampler). For analysis an acetonitrile (A)/0.1M triethylammonium acetate gradient was used: 5% to 35% A from 0 to 10 min, then 35% to 40% A from 10 to 20 min, then 40% to 95% A from 20 to 25 min, flow rate=1.0 mL/min/50% A from 8 to 9 min, 9 to 26 min at 50% flow rate=1.0 mL/min, t_{R}(DMT-off) 10-11 min, t_{R}(DMT-on) 1416 min. The DMT-on fraction was collected and was evaporated in vacuum, redissolved in water and the DMT group was removed as described below.

Dedimethoxytritylation

An aliquot (30 μL) was transferred into an Eppendorff tube (1.5 mL), and acetic acid (50%, 30 μL) was added. After 30 min at room temperature, sodium acetate (2.5M, 20 μL) was added, followed by cold ethanol (1.2 mL). The mixture was vortexed and cooled in dry ice for 20 min. The precipitate was spun down on a centrifuge, the supernatant was discarded and the precipitate was rinsed with ethanol and dried under vacuum.

ES/MS Sample Preparation

HPLC-purified and dedimethoxytritylated oligonucleotide was dissolved in 50 μL water, ammonium acetate (10 M, 5 μL) and ethanol were added and vortexed. The mixture was cooled in dry ice for 20 min and after centrifugation the precipitate was isolated. This procedure was repeated two more times to convert the oligonucleotide to the ammonium form. The oligonucleotide was redissolved in water/iso-propanol (1:1, 300 μL) and piperidine (10 μL) was added.

What is claimed is:

1. An oligomeric compound having the formula: 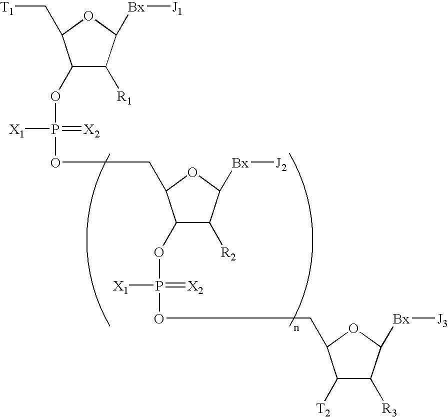
wherein:
each Bx is, independently, a heterocyclic base moiety;
J₁, J₃ and each J₂ is, independently, hydrogen or a modified phosphate group having the structure: 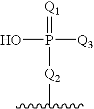
wherein
one of Q₁ and Q₂ is S and the other of Q₁ and Q₂ is O;
Q₃ is OH or CH₃;
R₁, R₃ and each R₂ is, independently, hydrogen, hydroxyl, a sugar substituent group a protected sugar substituent group or said modified phosphate group;
each T₁ and T₂ is, independently, hydroxyl, a protected hydroxyl, an oligonucleotide, an oligonucleoside or said modified phosphate group;
each X₁ and X₂ is, independently, O or S wherein at least one X₁ is S;
n is from 3 to 48; and
wherein at least one of J₁, J₂, J₃, T₁ or T₂ is said modified phosphate group.

2. The oligomeric compound of claim 1 wherein Q₁ is S.

3. The oligomeric compound of claim 1 wherein Q₂ is S.

4. The oligomeric compound of claim 1 wherein Q₃ is CH₃.

5. The oligomeric compound of claim 1 wherein J₁ is said modified phosphate group.

6. The oligomeric compound of claim 1 wherein at least one J₂ is said modified phosphate group.

7. The oligomeric compound of claim 1 wherein J₃ is said modified phosphate group.

8. The oligomeric compound of claim 1 wherein R₁ is a modified phosphate group.

9. The oligomeric compound of claim 1 wherein at least one R₂ is a modified phosphate group.

10. The oligomeric compound of claim 1 wherein R₃ is a modified phosphate group.

11. The oligomeric compound of claim 1 wherein R₁, R₃ and each R₂ is hydrogen.

12. The oligomeric compound of claim 1 wherein R₁, R₃ and each R₂ is hydroxyl.

13. The oligomeric compound of claim 1 wherein R₁, R₃ and each R₂ is hydrogen, hydroxyl a sugar substituent group or a protected sugar substituent group.

14. The oligomeric compound of claim 1 wherein at least one of R₁, R₂ or R₃ is an optionally protected sugar substituent group.

15. The oligomeric compound of claim 1 wherein each X₂ is S.

16. The oligomeric compound of claim 1 wherein each heterocyclic base moiety is, independently, adenine, cytosine, 5-methylcytosine, thymine, uracil, guanine or 2-aminoadenine.

17. The oligomeric compound of claim 1 wherein n is from about 8 to about 30.

18. The oligomeric compound of claim 1 wherein n is from about 15 to 25.

19. A method of treating an organism having a disease characterized by the undesired production of a protein comprising contacting the organism with an oligomeric compound of claim 1.

20. A pharmaceutical composition comprising:
a pharmaceutically effective amount of an oligomeric compound of claim 1; and
a pharmaceutically acceptable diluent or carrier.

21. A method of modifying in vitro a nucleic acid, comprising contacting a test solution containing RNase H and said nucleic acid with an oligomeric compound of claim 1.

22. A method of concurrently enhancing hybridization and RNase H activation in a organism comprising contacting the organism with an oligomeric compound of claim 1.

23. A method comprising contacting a cell with an oligomeric compound of claim 1.

24. An oligomeric compound having the formula: 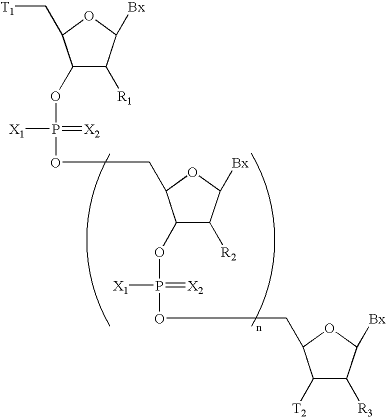
wherein
each Bx is, independently, a heterocyclic base moiety;
each T₁ and T₂ is, independently, hydroxyl, a protected hydroxyl, an oligonucleotide, an oligonucleoside or a modified phosphate group having the formula; 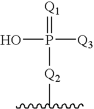
wherein
one of Q₁ and Q₂ is S and the other of Q₁ and Q₂ is O;
Q₃ is OH or CH₃;
R₁, R₃ and each R₂ is, independently, hydrogen, hydroxyl, a sugar substituent groups or a protected sugar substituent group;
each X₁ and X₂ is, independently, O or S wherein at least one X₁ is S; and
n is from 3 to 48;
wherein at least one of X₁, X₂, J₁, J₂ and J₃ is said modified phosphate group.

25. The oligomeric compound of claim 24 wherein Q₁ is S.

26. The oligomeric compound of claim 24 wherein Q₂ is S.

27. The oligomeric compound of claim 24 wherein Q₃ is CH₃.

28. The oligomeric compound of claim 24 wherein J₁ is said modified phosphate group.

29. The oligomeric compound of claim 24 wherein at least one J₂ is a modified phosphate group.

30. The oligomeric compound of claim 24 wherein J₃ is said modified phosphate group.

31. The oligomeric compound of claim 24 wherein R₁ is a modified phosphate group.

32. The oligomeric compound of claim 24 wherein at least one R₂ is a modified phosphate group.

33. The oligomeric compound of claim 24 wherein R₃ is a modified phosphate group.

34. The oligomeric compound of claim 24 wherein R₁, R₃ and each R₂ is hydrogen.

35. The oligomeric compound of claim 24 wherein R₁, R₃ and each R₂ is hydroxyl.

36. The oligomeric compound of claim 24 wherein R₁, R₃ and each R₂ is hydrogen, hydroxyl a sugar substituent group or a protected sugar substituent group.

37. The oligomeric compound of claim 24 wherein at least one of R₁, R₂ or R₃ is an optionally protected sugar substituent group.

38. The oligomeric compound of claim 24 wherein each X₂ is S.

39. The oligomeric compound of claim 24 wherein each heterocyclic base moiety is, independently, adenine, cytosine, 5-methylcytosine, thymine, uracil, guanine or 2-aminoadenine.

40. The oligomeric compound of claim 24 wherein n is from about 8 to about 30.

41. The oligomeric compound of claim 24 wherein n is from about 15 to 25.


SEQUENCE LISTING (66 sequences)

SEQ ID NO: 1 - DNA, 20 bp, Artificial
  Synthetic Construct
gcccaagctggcatccgtca

SEQ ID NO: 2 - DNA, 19 bp, Artificial
  Synthetic Construct
gcccaagctggcatccgtc

SEQ ID NO: 3 - DNA, 20 bp, Artificial
  Synthetic Construct
cgggttcgaccgtaggcagt

SEQ ID NO: 4 - RNA, 20 nt, Artificial
  Oligonucleotide
uuugucucugguccuuacuu

SEQ ID NO: 5 - RNA, 20 nt, Artificial
  Oligonucleotide
aaacagagaccaggaaugaa

SEQ ID NO: 6 - RNA, 20 nt, Artificial
  Oligonucleotide
uuugucucugguccuuacuu

SEQ ID NO: 7 - RNA, 20 nt, Artificial
  Oligonucleotide
uuugucucugguccuuacuu

SEQ ID NO: 8 - RNA, 20 nt, Artificial
  Oligonucleotide
aaacagagaccaggaaugaa

SEQ ID NO: 9 - RNA, 20 nt, Artificial
  Oligonucleotide
uuugucucugguccuuacuu

SEQ ID NO: 10 - RNA, 20 nt, Artificial
  Oligonucleotide
uuugucucugguccuuacuu

SEQ ID NO: 11 - RNA, 20 nt, Artificial
  Oligonucleotide
aaacagagaccaggaaugaa

SEQ ID NO: 12 - RNA, 20 nt, Artificial
  Oligonucleotide
uuugucucugguccuuacuu

SEQ ID NO: 13 - RNA, 20 nt, Artificial
  Oligonucleotide
uuugucucugguccuuacuu

SEQ ID NO: 14 - RNA, 20 nt, Artificial
  Oligonucleotide
aaacagagaccaggaaugaa

SEQ ID NO: 15 - RNA, 20 nt, Artificial
  Oligonucleotide
uuugucucugguccuuacuu

SEQ ID NO: 16 - RNA, 20 nt, Artificial
  oligonucleotide
uuugucucugguccuuacuu

SEQ ID NO: 17 - RNA, 20 nt, Artificial
  Oligonucleotide
aaacagagaccaggaaugaa

SEQ ID NO: 18 - RNA, 20 nt, Artificial
  Oligonucleotide
uuugucucugguccuuacuu

SEQ ID NO: 19 - RNA, 20 nt, Artificial
  Oligonucleotide
uuugucucugguccuuacuu

SEQ ID NO: 20 - RNA, 20 nt, Artificial
  Oligonucleotide
aaacagagaccaggaaugaa

SEQ ID NO: 21 - RNA, 20 nt, Artificial
  Oligonucleotide
uuugucucugguccuuacuu

SEQ ID NO: 22 - RNA, 20 nt, Artificial
  Oligonucleotide
uuugucucugguccuuacuu

SEQ ID NO: 23 - RNA, 20 nt, Artificial
  Oligonucleotide
aaacagagaccaggaaugaa

SEQ ID NO: 24 - RNA, 20 nt, Artificial
  Oligonucleotide
uuugucucugguccuuacuu

SEQ ID NO: 25 - RNA, 20 nt, Artificial
  Oligonucleotide
uuugucucugguccuuacuu

SEQ ID NO: 26 - RNA, 20 nt, Artificial
  Oligonucleotide
aaacagagaccaggaaugaa

SEQ ID NO: 27 - RNA, 20 nt, Artificial
  Oligonucleotide
uuugucucugguccuuacuu

SEQ ID NO: 28 - RNA, 20 nt, Artificial
  Oligonucleotide
uuugucucugguccuuacuu

SEQ ID NO: 29 - RNA, 20 nt, Artificial
  Oligonucleotide
aaacagagaccaggaaugaa

SEQ ID NO: 30 - RNA, 20 nt, Artificial
  Oligonucleotide
uuugucucugguccuuacuu

SEQ ID NO: 31 - RNA, 20 nt, Artificial
  Oligonucleotide
uuugucucugguccuuacuu

SEQ ID NO: 32 - RNA, 20 nt, Artificial
  Oligonucleotide
aaacagagaccaggaaugaa

SEQ ID NO: 33 - RNA, 20 nt, Artificial
  Oligonucleotide
uuugucucugguccuuacuu

SEQ ID NO: 34 - RNA, 20 nt, Artificial
  Oligonucleotide
uuugucucugguccuuacuu

SEQ ID NO: 35 - RNA, 20 nt, Artificial
  Oligonucleotide
aaacagagaccaggaaugaa

SEQ ID NO: 36 - RNA, 20 nt, Artificial
  Oligonucleotide
uuugucucugguccuuacuu

SEQ ID NO: 37 - RNA, 20 nt, Artificial
  Oligonucleotide
uuugucucugguccuuacuu

SEQ ID NO: 38 - RNA, 20 nt, Artificial
  Oligonucleotide
aaacagagaccaggaaugaa

SEQ ID NO: 39 - RNA, 20 nt, Artificial
  Oligonucleotide
uuugucucugguccuuacuu

SEQ ID NO: 40 - RNA, 20 nt, Artificial
  Oligonucleotide
uuugucucugguccuuacuu

SEQ ID NO: 41 - RNA, 20 nt, Artificial
  Oligonucleotide
aaacagagaccaggaaugaa

SEQ ID NO: 42 - RNA, 20 nt, Artificial
  Oligonucleotide
uuugucucugguccuuacuu

SEQ ID NO: 43 - RNA, 20 nt, Artificial
  Oligonucleotide
uuugucucugguccuuacuu

SEQ ID NO: 44 - RNA, 20 nt, Artificial
  Oligonucleotide
aaacagagaccaggaaugaa

SEQ ID NO: 45 - RNA, 20 nt, Artificial
  Oligonucleodide
uuugucucugguccuuacuu

SEQ ID NO: 46 - RNA, 20 nt, Artificial
  Oligonucleotide
uuugucucugguccuuacuu

SEQ ID NO: 47 - RNA, 20 nt, Artificial
  Oligonucleotide
aaacagagaccaggaaugaa

SEQ ID NO: 48 - RNA, 20 nt, Artificial
  Oligonucleotide
uuugucucugguccuuacuu

SEQ ID NO: 49 - RNA, 20 nt, Artificial
  Oligonucleotide
uuugucucugguccuuacuu

SEQ ID NO: 50 - RNA, 20 nt, Artificial
  Oligonucleotide
aaacagagaccaggaaugaa

SEQ ID NO: 51 - RNA, 20 nt, Artificial
  Oligonucleotide
uuugucucugguccuuacuu

SEQ ID NO: 52 - DNA, 20 bp, Artificial
  Oligonucleotide
tccgtcatcgctcctcaggg

SEQ ID NO: 53 - DNA, 20 bp, Artificial
  Oligonucleotide
gtgcgcgcgagcccgaaatc

SEQ ID NO: 54 - DNA, 20 bp, Artificial
  Oligonucleotide
atgcattctgcccccaagga

SEQ ID NO: 55 - DNA, 19 bp, Artificial
  Oligonucleotide
ctacgctttccacgcacag

SEQ ID NO: 56 - DNA, 19 bp, Artificial
  Oligonucleotide
ctacgctttccacgcacag

SEQ ID NO: 57 - DNA, 19 bp, Artificial
  Oligonucleotide
ctacgctttccacgcacag

SEQ ID NO: 58 - DNA, 19 bp, Artificial
  Oligonucleotide
ctacgctttccacgcacag

SEQ ID NO: 59 - DNA, 19 bp, Artificial
  Oligonucleotide
ctacgctttccacgcacag

SEQ ID NO: 60 - DNA, 19 bp, Artificial
  Oligonucleotide
ctacgctttccacgcacag

SEQ ID NO: 61 - DNA, 19 bp, Artificial
  Oligonucleotide
ctacgctttccacgcacag

SEQ ID NO: 62 - DNA, 19 bp, Artificial
  Oligonucleotide
ctacgctttccacgcacag

SEQ ID NO: 63 - DNA, 19 bp, Artificial
  Oligonucleotide
ctacgctttccacgcacag

SEQ ID NO: 64 - DNA, 19 bp, Artificial
  Oligonucleotide
cgagaggcggacgggaccg

SEQ ID NO: 65 - DNA, 21 bp, Artificial
  Oligonucleotide
cgagaggcggacgggaccgtt

SEQ ID NO: 66 - DNA, 21 bp, Artificial
  Oligonucleotide
cggtcccgtccgcctctcgtt